(19) United States
(12) Patent Application Publication
(10) Pub. No.: US 2013/0102596 A1
(43) Pub. Date: Apr. 25, 2013

(54) METHOD AND DEVICE FOR ADMINISTERING XINAFOATE SALT OF N4-(2,2-DIFLUORO-4H-BENZO [1,4]OXAZIN-3-ONE)-6-YL]-5-FLUORO-N2-[3- (METHYLAMINOCARBONYLMETHYLENEOXY) PHENYL]2,4-PYRIMIDINEDIAMINE

(71) Applicant: RIGEL PHARMACEUTICALS, INC., South San Francisco, CA (US)

(72) Inventor: Thomas Sun, Palo Alto, CA (US)

(73) Assignee: RIGEL PHARMACEUTICALS, INC., South San Francisco, CA (US)

(21) Appl. No.: 13/656,395

(22) Filed: Oct. 19, 2012

Related U.S. Application Data
(60) Provisional application No. 61/550,235, filed on Oct. 21, 2011.

(51) Int. Cl. A61K 31/536; A61M 15/00; B65B 1/04; A61P 11/00

(52) U.S. Cl. 514/230.5; 128/203.12; 53/410

(57) ABSTRACT

Disclosed embodiments concern a device for administering a xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, and a method for making and using the device. Particular disclosed embodiments concern formulating the xinafoate salt for administration via the device.

### CROSS-REFERENCE TO RELATED APPLICATION

This claims the benefit of U.S. Provisional Application No. 61/550,235, filed Oct. 21, 2011, which is incorporated in its entirety herein by reference.

### FIELD

Disclosed embodiments concern N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, pharmaceutically acceptable salts thereof, particularly xinafoate salts, and compositions comprising the pyrimidinediamine or salt thereof, and embodiments of a device and method for administering such compounds or compositions.

### BACKGROUND

In order for a compound to be developed as a drug, it is desirable to obtain a form of that compound (commonly referred to as a drug substance) that is stable and does not degrade on storage, and that can be reliably prepared and purified on a large scale. Such characteristics may be found in a drug which is crystalline and has a high melting point. High-melting point crystalline solids may be purified by re-crystallization and are stable during storage. Furthermore, the drug substance should be suitable for formulation in a dosage form chosen according to the intended route of administration. For example, non-hygroscopicity is a property of particular interest to as formulating dry powders suitable for inhalation. Compatibility with conventional excipients is a further characteristic of interest. Furthermore, the drug substance usually will undergo processing in order to achieve a particle size suitable for inhalation and any crystalline form should be stable during such processing so that the properties of the final product are predictable and reliable. Thus, in many instances, whether a compound is suitable for commercialization as a drug depends on preparing a form of the compound having a unique combination of properties determined according to the intended route of administration.

### SUMMARY

Disclosed embodiments concern a device and method for administering a xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions comprising the salt. A variety of embodiments of the device are disclosed herein, all of which are suitable for administering the disclosed xinafoate salt neat or as a composition. The device may comprise a variety of operatively associated components to provide the ability to administer various forms of the xinafoate salt.

In particular disclosed embodiments, the device comprises a housing and a source of the disclosed xinafoate salt, or compositions thereof. The housing typically is configured to be held in a patient's hand and may comprise a patient interface, such as a mouthpiece or a nasal adapter, in communication with the source of the xinafoate salt or compositions thereof. In particular disclosed embodiments, the housing may further comprise a dispensing mechanism, such as a plunger, a push-button, an impactor, an impeller, and combinations thereof, as well as a reset mechanism to reset the dispensing mechanism. The dispensing mechanism may be used in combination with an aerosolization element, which provides the ability to aerosolize the disclosed xinafoate salt, or compositions thereof, for facile delivery to the patient using the device. The aerosolization element may comprise a chamber through which gas flows to aerosolize the xinafoate salt, or compositions thereof. Certain disclosed embodiments of the aerosolization element include a gas circulation mechanism comprising an impeller or an impactor. In other particular disclosed embodiments, the housing may comprise a triggering mechanism and a reset component, the triggering mechanism comprising a vane and an activator component. In particular disclosed embodiments, the triggering mechanism may further comprise a rocker and a catch that interengage with each other as well as will the vane.

The device may be used in conjunction with a variety of different sources of the xinafoate salt, or compositions thereof. The source may be one that holds the xinafoate salt (or composition thereof) as a dry powder, such as a powder reservoir, and can be detachable or integrated with the device. In certain disclosed embodiments, the source may be a blister package comprising one or more blisters containing the xinafoate salt, or compositions thereof. Also contemplated as sources are elongate carriers loaded with the xinafoate salt, or compositions thereof. Particular embodiments concern a microstructured tape that may be used to sequentially release a number of dosages to the patient using the device. The microstructured carrier tape comprises at least one and more typically, plural microdepressions, such as microgrooves, microdimples, microblisters, and combinations thereof, to hold the xinafoate salt, and/or compositions thereof.

Disclosed embodiments also concern administering the disclosed xinafoate salt neat (i.e., excipient-free) or as a composition. The composition may comprise the xinafoate salt and a pharmaceutically acceptable carrier, which may be a carbohydrate selected from lactose, dextran, glucose, maltose, sorbitol, xylitol, fructose, sucrose, trehalose, and combinations thereof. In certain disclosed embodiments, the lactose is either lactose monohydrate or anhydrous lactose. Compositions of the xinafoate salt typically comprise about 1 to about 20 weight percent of the xinafoate salt and from about 99 to about 80 weight percent of a pharmaceutically acceptable carrier. A person of ordinary skill in the art will recognize that these amounts are based on the active pharmaceutical ingredient free base. The xinafoate salt has certain chemical and physical properties that contribute to its ability to be delivered using the disclosed device. For example, the disclosed xinafoate salt typically is not hygroscopic, and does not form hydrates or solvates. Furthermore, certain disclosed embodiments of the composition are stable in conditions ranging from 25° C/60% relative humidity to about 40° C/75% relative humidity. Thus, administering the salt may include administering a non-hygroscopic xinafoate salt, a non-hydrated xinafoate salt, a non-solvated xinafoate salt, or combinations thereof

Also disclosed herein is a method for making the disclosed xinafoate salt and a method for formulating it so that it may be delivered to the patient using the disclosed device. Also disclosed herein are embodiments concerning a method for making and using an inhaler and a method for associating the source of the xinafoate salt, or compositions thereof, with the disclosed inhaler.

The foregoing and other objects, features, and advantages of the invention will become more apparent from the following detailed description, which proceeds with reference to the accompanying figures.

### BRIEF DESCRIPTION OF THE DRAWINGS

FIG. 1 is a side view of a dry powder reservoir.

FIG. 2 is a sectional view of a dosage chamber in communication with a pressure chamber and a powder reservoir.

FIG. 3 is a top view of a plurality of blisters of a blister package disposed on a disk.

FIG. 4 is a top view of a plurality of blisters of a blister package disposed in a row.

FIG. 5 is a side elevation view of a plurality of blisters of a blister package disposed in a cylinder.

FIG. 6 is a side elevation view of a plurality of blisters of a blister package disposed in a hexagon.

FIG. 7 is a sectional view of an elongate carrier disposed in a cassette.

FIG. 8 is a sectional view of an embodiment of a housing.

FIG. 9 is a side view of an embodiment of a housing.

FIG. 10 is a sectional view of a housing showing a moveable cover and internal linkage mechanism.

FIG. 11 is a front elevation view of an embodiment of a housing with a base, a dosage preparation section, and a patient interface.

FIG. 12 is a perspective view of an embodiment of a housing having a round profile.

FIG. 13 is a sectional view of a chamber in communication with a patient interface.

FIG. 14 is a sectional view of an embodiment of a housing containing a spring and plunger, a chamber, and a nozzle.

FIG. 15 is a sectional view of a chamber with gas conduits leading into and out of the chamber.

FIG. 16 is a sectional view of a chamber with air conduits in fluid communication with inlets.

FIG. 17 is a plan view of an embodiment of an impeller.

FIG. 18 is a sectional view of a housing containing an elongate carrier disposed on spools and a striking hammer.

FIG. 19 is a sectional view of a particular disclosed embodiment of the device and its components while not in use.

FIG. 20 is a sectional view of a particular disclosed embodiment of the device when its components are activated.

FIG. 21 is a sectional view of a particular disclosed embodiment of the device illustrating the components in the appropriate positions for dispensing the xinafoate salt, or compositions thereof.

FIG. 22 is an image of a particular disclosed embodiment for filling microdepressions wherein an elongate carrier is prepared with the xinafoate salt, or compositions thereof.

FIG. 23 is an image of a trace obtained using differential scanning calorimetry illustrating a sharp endothermic melting peak from the disclosed xinafoate salt.

FIG. 24 is an image of the pattern obtained from the disclosed xinafoate salt using powder X-ray diffraction analysis.

FIG. 25 is an image of a simulated pattern obtained from single crystal X-ray analysis.

FIG. 26 is an image of a spectrum obtained from Fourier Transform Infra-red (FT-IR) analysis of the disclosed xinafoate salt.

FIG. 27 is an expanded image of the spectrum in FIG. 26, which illustrates the fingerprint region of the spectrum.

FIG. 28 is an image of a spectrum obtained from Fourier Transform Raman spectroscopic analysis of the disclosed xinafoate salt.

FIG. 29 is an expanded image of the spectrum in FIG. 28, which illustrates the fingerprint region of the spectrum.

FIG. 30 is an image of the spectrum obtained from proton decoupled ¹³C solid state nuclear magnetic resonance (NMR) spectroscopic analysis of the disclosed xinafoate salt.

FIG. 31 is an image of the spectrum obtained from fluorine solid state NMR analysis of the disclosed xinafoate salt.

### DETAILED DESCRIPTION

### I. Terms and Abbreviations

Unless otherwise noted, technical terms are used according to conventional usage. As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all molecular weight or molecular mass values, given compounds are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various examples of this disclosure, the following explanations of specific teems are provided:

“Pharmaceutically effective amount” and “therapeutically effective amount” refer to an amount of a compound sufficient to treat a specified disorder or disease or one or more of its symptoms and/or to prevent the occurrence of the disease or disorder. The amount of a compound which constitutes a “therapeutically effective amount” will vary depending on the compound, the disease state and its severity, the age of the patient to be treated, and the like. The therapeutically effective amount can be determined routinely by one of ordinary skill in the art.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, for example, arresting or slowing its development;

(iii) relieving the disease or condition, for example, causing regression of the disease or condition or a symptom thereof; or

(iv) stabilizing the disease or condition.

As used herein, the terms “disease” and “condition” can be used interchangeably or can be different in that the particular malady or condition may not have a known causative agent (so that etiology has not yet been worked out) and it is therefore not yet recognized as a disease but only as an undesirable condition or syndrome, where a more or less specific set of symptoms have been identified by clinicians.

### II. Device Components Generally

Disclosed embodiments concern a device for delivering the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, to one or more patients in single dosage applications, or for multiple administrations. In certain disclosed embodiments, the device may be an inhaler, such as, but not limited to, a dry powder inhaler.

Particular disclosed embodiments concern a device comprising a housing capable of substantially encompassing one or more components of the device that is typically shaped to be held in a patient's hand. The device may further comprise a patient interface selected from those suitable for administering a dose of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, to a patient. In some disclosed embodiments, the patient interface may be designed for delivering the dose to a patient's mouth (e.g. a mouthpiece) and in other disclosed embodiments the patient interface may be designed to deliver the dose to a patient's nose (e.g. a nasal adapter).

For certain embodiments, the housing integrates a source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, that is integral with and not detachable from the housing. In other disclosed embodiments, the housing includes a detachable source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine or compositions thereof. Certain disclosed embodiments concern a device wherein the housing comprises a patient interface coupled to a detachable source of the xinafoate salt via an interface component comprising an aerosolization element.

The housing may further comprise a moveable cover attached to the housing in a manner that allows the moveable cover to be positioned over or away from the patient interface. Opening the moveable cover facilitates a variety of device functions including, but not limited to, exposing the patient to the xinafoate salt or composition thereof, loading a predetermined dose of the disclosed xinafoate salt into an aerosolization element, readying a dispensing mechanism, detaching a xinafoate salt source from the housing, or any combinations thereof. In particular disclosed embodiments, the housing may comprise a dosage preparation component and a storage base. The dosage preparation component, in this context, may have a flat surface upon which a blister pack may be placed in preparation for dispensing the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine.

The housing may be operatively associated with a dispensing mechanism of any type suitable for dispensing the xinafoate salt through the patient interface. In particular disclosed embodiments, the dispensing mechanism may be selected from a plunger, a push-button, an impactor, an impeller and combinations thereof. The housing may further comprise a reset mechanism to reset the dispensing mechanism after an initial use for a subsequent use. The reset mechanism may be a projection on the linkage of the moveable cover, such that the device is reset for a subsequent use when the cover is closed after the patient has used the device for a first use.

The housing may further comprise an advancement mechanism to facilitate delivery of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. The advancement mechanism may sequentially deliver dosages of the disclosed xinafoate salt of N4-[(2,2-difluoro-4H-benzo[1,4]oxazin-3-one)-6-yl]-5-fluoro-N243-(methylaminocarbonylmethyleneoxy)phenyl]-2,4-pyrimidinediamine, or compositions thereof, to a patient. In certain disclosed embodiments, the advancement mechanism advances a dose to the dispensing mechanism and/or aerosolization element. In particular disclosed embodiments, the advancement mechanism may be used in combination with an elongate carrier (e.g. a microstructured carrier tape). For microstructured carrier tapes, the advancement mechanism may comprise a rotatable winding spool. Rotation of the winding spool unwinds the elongate carrier (e.g. microstructured carrier tape), which is wound onto a winding spool.

Also contemplated in this disclosure is a housing that comprises one or more sensors and/or microprocessors capable of detecting a pressure change induced by the inhalation of a patient through the patient interface. Detecting the pressure change may actuate the aerosolization element to aerosolize the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, and deliver it to the patient through the patient interface.

Particular embodiments of the disclosed device comprise an aerosolization element capable of converting a condensed or agglomerated form of the xinafoate salt of N4- [(2,2-difluoro-4H-benzo [1,4]oxazin-3-one)-6-yl]-5-fluoro-N2-[3-(methylaminocarbonylmethyleneoxy)phenyl]-2,4-pyrimidinediamine, or compositions thereof, to deagglomerated free particle form. In certain disclosed embodiments, the aerosolization element may comprise a chamber and/or a nozzle through which the aerosolized dose of the xinafoate salt, or compositions thereof, may flow. Aerosolization may be facilitated using a source of air or gas that allows the deagglomerated salt to travel from the device to the patient. In certain disclosed embodiments, a patient using the device may provide the air source, such as when the patient inhales, thus providing air flow through the device.

In particular embodiments, a chamber may be configured to accept the flow of the air or gas used to deliver a deagglomerated dose. The chamber may be configured to ensure that aerosolized particles are of a sufficiently small diameter to facilitate absorption in the lungs of a patient. Particular disclosed embodiments concern an aerosolization element comprising one or more impactors or impellers. The impeller may be part of a gas circulation mechanism that promotes gas flow throughout the device.

In particular disclosed embodiments, the device may comprise an inhalation-activatable triggering mechanism that controls dispensation from the source of the xinafoate salt. In particular disclosed embodiments, the triggering mechanism actuates the device, thus obviating the need for handling co-ordination by the patient. The device may further comprise a reset component. In particular disclosed embodiments, the triggering mechanism comprises a vane capable of pivotal movement between a closed position and an open position. In certain disclosed embodiments, the vane is positioned such that inhalation through the patient interface generates an air flow that effectuates the vane's pivotal movement. The vane pivot point is typically positioned towards one end of the vane. Particular disclosed embodiments concern a device that further comprises an activator component that moves between a restrained position and a dispensing position during use. The activator component's movement controls dispensing of the xinafoate salt from the source, and typically, the activator component is biased towards the dispensing position.

In certain disclosed embodiments, the triggering mechanism may be arranged such that when the activator component is in a restrained position and the vane is closed, the vane mechanically blocks the activator component from moving from its restrained position. This mechanical blocking may be effectuated by one or more movable intermediate components whose movements to release the mechanical blocking action are controlled by the vane. When the vane pivots from a closed position to an open position the mechanical blocking action is removed, thus allowing movement of the activator component to allow dispensation of the xinafoate salt. In particular disclosed embodiments, the reset component causes the activator component to move back into its restrained position, which directly or indirectly via one or more intermediate components causes the vane to move from a substantially open position to a closed position. A person of ordinary skill in the art will recognize that even though disclosed embodiments concern an activator component that is arranged to move pivotally, it is also capable of being arranged to move reciprocally or linearly.

In particular disclosed embodiments, the components of the triggering mechanism are arranged such that they may mechanically interengage during the reset cycle. For example by returning the activator component to a restrained position, the other components are returned to their respective positions ready for the next triggering sequence.

In certain disclosed embodiments, the vane is positioned within the patient interface and arranged such that it may be substantially returned to its closed position prior to initiating the reset cycle, providing the vane is positively engaged by a component of the triggering mechanism as the mechanism is reset. In particular disclosed embodiments, the blocking and reset component are positioned at an end of the vane near the pivot point. In certain disclosed embodiments, these components are introduced by using a vane comprising a projection. In particular disclosed embodiments, when the activator component is restrained and the vane is closed, the blocking surface (e.g. projection) mechanically engages the activator component, and when the vane is pivoted from its closed to open position, the blocking surface is moved out of mechanical engagement with the activator component, which allows it to dispense the xinafoate salt from its source. The reset component can then move the activator component from its dispensing position to its restrained position, which causes engagement of the reset surface by the activator component, thus pivoting the vane to its closed position and blocking the activator component in its restrained position.

In particular disclosed embodiments, the triggering mechanism may comprise a vane, catch and activator component. The catch may be pivotally mounted for movement between (1) a blocking position in which it mechanically prevents the activator component from moving from its restrained position, and (2) a release position in which it allows the activator component to dispense the xinafoate salt from its source. In particular disclosed embodiments, the catch and vane each having a respective engagable end to allow movement between the two. In certain disclosed embodiments, the catch also comprises a blocking surface to engage the activator component in its restrained position and a reset surface which is engaged by the activator component during movement from dispensing to its restrained position. Typically, the activator component is moved back to its restrained position by the reset component, which then causes the catch to move back to its blocking position, resulting in the vane being closed.

Particular disclosed embodiments concern a triggering mechanism comprising a vane, a rocker, a catch and an activator component. The catch is typically arranged as previously described, and the rocker is similarly mounted for pivotal movement. In particular disclosed embodiments, the rocker may comprise an end that is engagable with one end of the vane, allowing movement between the two, and a second end engagable with the catch, allowing movement between the two. The catch may also comprise a blocking surface to engage the activator component in its restrained position and a reset surface which is engaged by the activator component during movement from dispensing to its restrained position. Typically, the catch is actuated by the reset component, which causes the catch to move back to its blocking position, which then effects movement of the rocker, thereby closing the vane. In particular disclosed embodiments, a device comprising a triggering mechanism comprising a catch, a rocker, a vane, and an activator component readily allows the triggering mechanism to be fitted into available areas in the device given that the pivot points of the components need not be arranged linearly.

In particular disclosed embodiments, the reset component for the triggering mechanism preferably acts directly on the activator component and moves it into its restrained position. The reset component may be a projection on the moveable cover, such that the device is reset when the cover is closed after the patient has used the device. Exemplary devices are described in U.S. Patent No. 5,408,994, which is incorporated herein by reference.

### III. Source of the Xinafoate Salt of N4-[(2,2-Difluoro-4h-Benzo[1,4]Oxazin-3-One)-6-yl]-5-Fluoro-N2-[3-(Methylaminocarbonylmethyleneoxy)Phenyl]-2,4-Pyrimidinediamine, or Compositions Thereof

Particular disclosed embodiments concern a source of the xinafoate salt, or compositions thereof, which is capable of storing dosages for administration to a patient. The source of the disclosed xinafoate salt may take many forms, including but not limited to a replaceable refill unit, a xinafoate salt powder reservoir, a blister package, an elongate carrier, such as a microstructured tape, and combinations thereof.

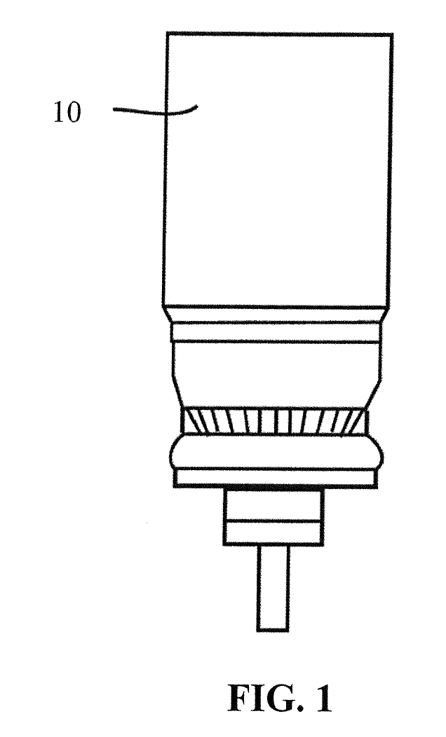

Particular disclosed embodiments concern a pressurized aerosol container, such as that illustrated in FIG. 1. FIG. 1 depicts a dry powder reservoir 10, which may be detachable from a housing of a delivery device. This allows the delivery device to be refilled with multiple replaceable sources of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. In certain disclosed embodiments the integral and/or detachable source 10 contains a predetermined amount of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof.

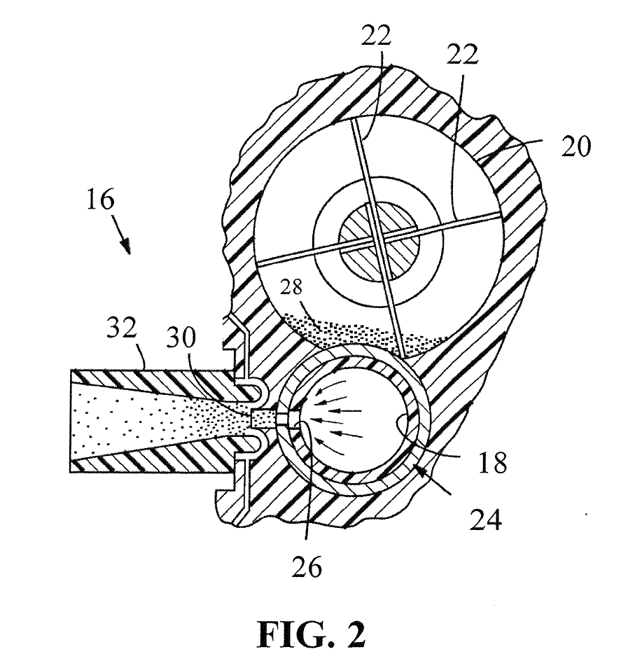

In particular disclosed embodiments, a source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, may be a powder reservoir 20, as illustrated in FIG. 2. The source may include loading members 22 comprising rotatable blades for packing a dose of the xinafoate salt (28) of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, into a dosage chamber 26 as disclosed in U.S. Pat. No. 6,119,688. In particular disclosed embodiments, the loading members 22 are made from a sufficiently flexible material such that when they contact the dosage element 24 as the powder reservoir 20 rotates, they deflect, ensuring that the dosage chamber 26 is consistently filled with a predetermined amount of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof.

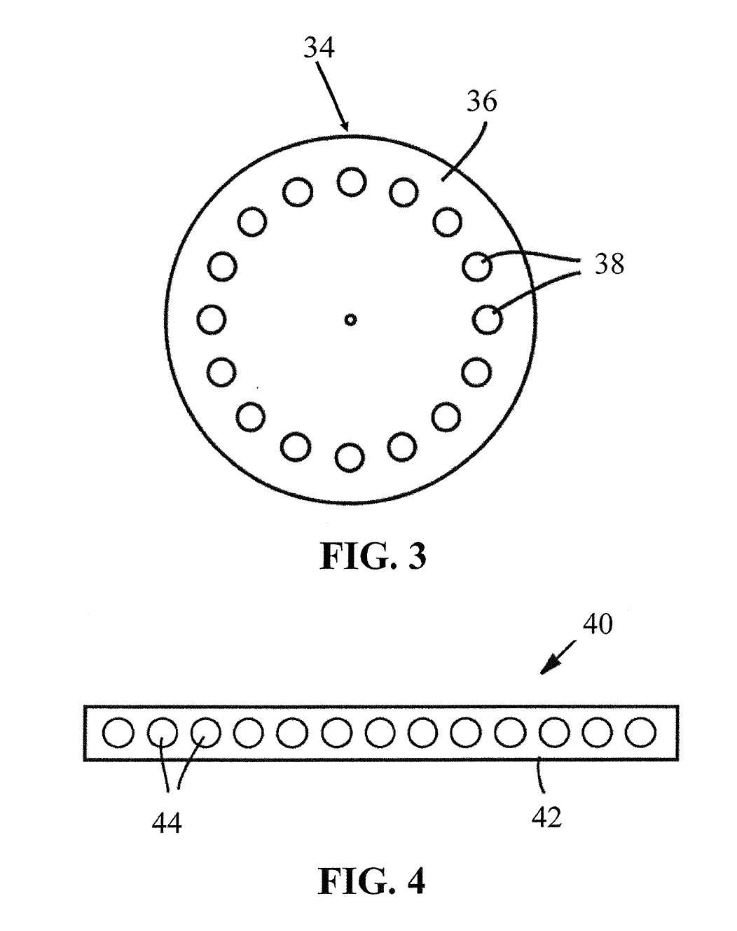

The disclosed xinafoate salt source may be a blister package, comprising one or more blisters containing a predetermined amount of xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. The blister package may have any geometric shape useful for dispensing the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. In certain disclosed embodiments, a blister package 34 may be a disk 36, as illustrated in FIG. 3. Disk 36 includes at least one, and typically a plurality of blisters 38, disposed around the disk. Disk 36 is rotated within the delivery device housing by an advancement mechanism, which acts to advance the next dose.

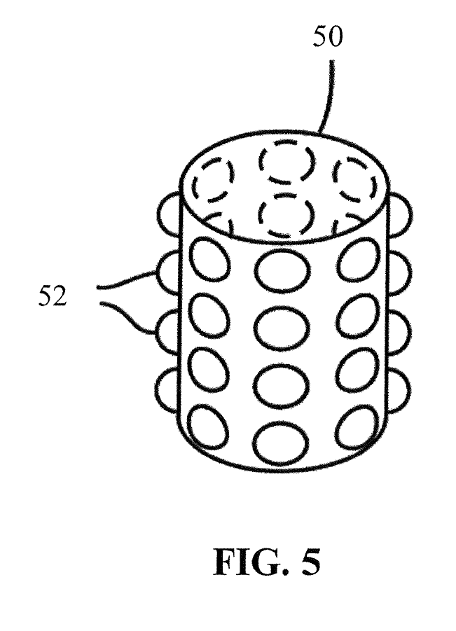

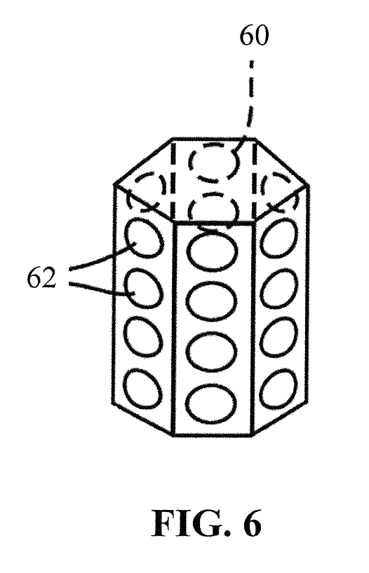

In other disclosed embodiments, the blister package may be a strip, comprising blisters in rows, such as the embodiment illustrated in FIG. 4. According to FIG. 4, the embodiment 40 comprises a strip 42 and a plurality of blisters 44. In other disclosed embodiments, the blister package may be a cylinder. FIG. 5 illustrates a cylindrical blister package 50 comprising a plurality of blisters 52. Other embodiments concern a blister package 60 having a hexagonal shape, such as that illustrated in FIG. 6, comprising a plurality of blisters 62.

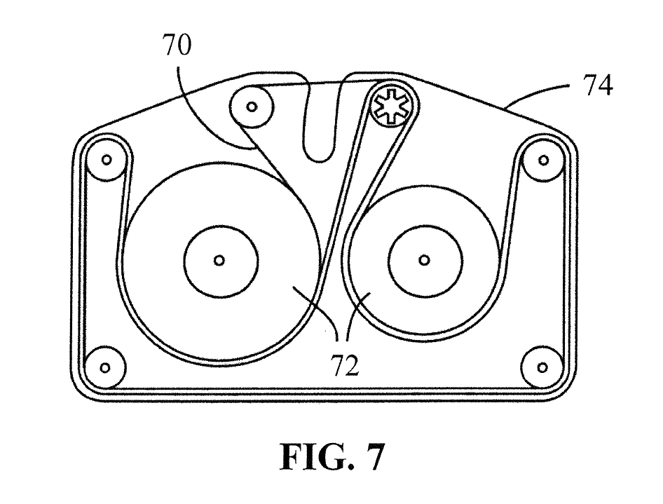

The disclosed source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, may also be an elongate carrier 70, such as a microstructured carrier tape or a cord, as shown in the device 74 of FIG. 7. Certain embodiments of a suitable microstructured carrier tape are disclosed in U.S. Pat. No. 5,619,984. In particular disclosed embodiments, the carrier 70 may be disposed on spools 72, such that the spent carrier tape or cord may be wound about one spool and fresh carrier may be advanced from the other. In particular embodiments, the device may comprise a tensioning element for holding an exposed portion of the elongate carrier taut. The elongate carrier may have microdepressions in which xinafoate salt is agglomerated for storage within the elongate carrier. In particular disclosed embodiments, the microdepressions may be selected from microgrooves, microdimples, microblisters, and combinations thereof. In certain disclosed embodiments, the entire assembly may be disposed in an enclosure such that it comprises a cassette 74, which may be attached and detached from a delivery device housing, thus enabling use of consecutive cassettes with the same delivery device.

### IV. Particular Embodiments

Particular disclosed embodiments may comprise any of the disclosed components in any combination suitable for administering the xinafoate salt, as illustrated in FIGS. 2 and 8-18. FIG. 2 illustrates a device 16 comprising an aerosolization element 18. A powder reservoir 20 communicates with a cylindrical dosage element 24. Dosage element 24 comprises a dosage chamber 26. Rotating chamber 26 causes a predetermined amount of the disclosed xinafoate salt (28) of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, to be transferred from the powder reservoir 20 to the breech 30 of a patient interface 32, as disclosed in U.S. Pat. No. 6,119,688, which is incorporated herein by reference. The aerosolization element 18 then delivers compressed gas at a sufficiently high pressure that the powdered xinafoate salt 28 is aerosolized and deagglomerated in the turbulent flow induced in the breech 30 of the patient interface 32. This particular disclosed embodiment of an aerosolization element specifically enables using the a powder reservoir; however, a person of ordinary skill in the art will recognize that it may be configured to accept other forms of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, as well.

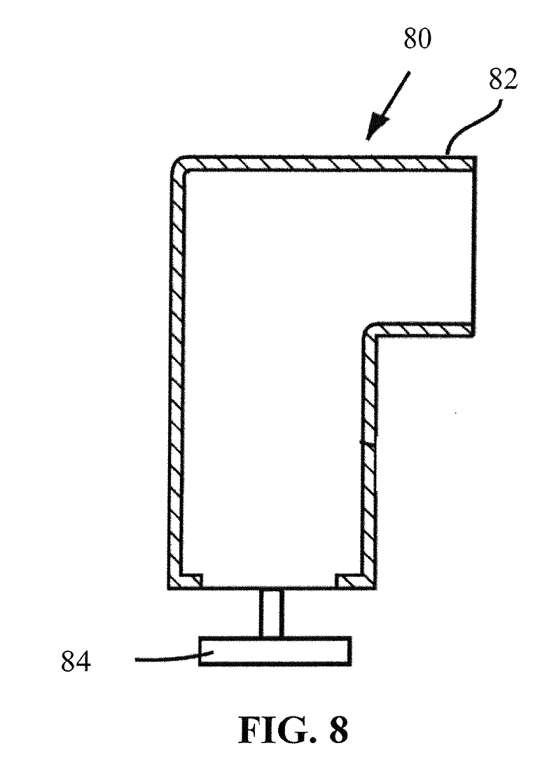

FIG. 8 is a schematic diagram illustrating a housing 80 having a patient interface 82. For this embodiment, patient interface 82 is a mouthpiece, as disclosed in U.S. Pat. No. 7,841,338, which is incorporated herein by reference. Housing 80 includes an actuatable dispensing mechanism 84 for dispensing a predetermined quantity of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof.

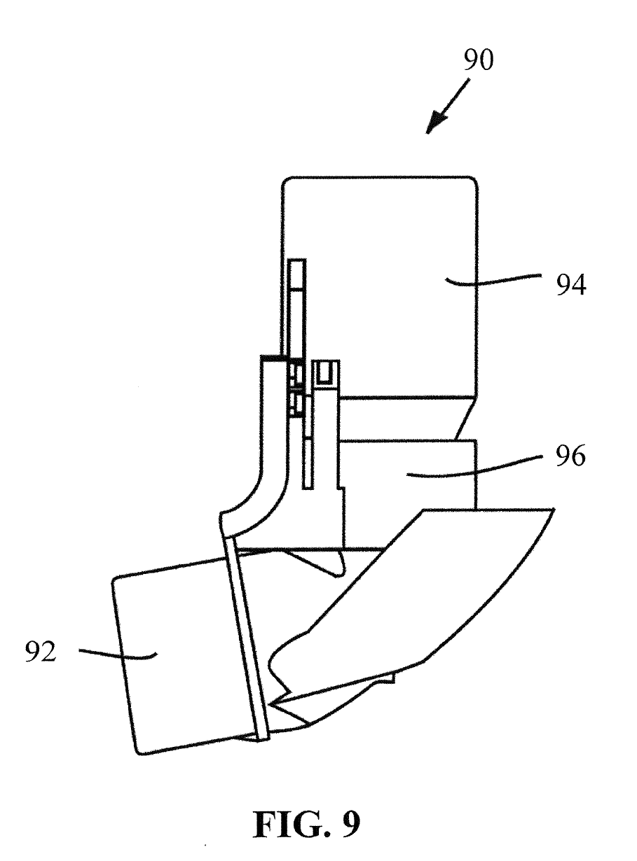

An additional embodiment of a device for administering the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, is illustrated in FIG. 9. FIG. 9 is a schematic diagram depicting a housing 90 as disclosed in U.S. Design Pat. No. D449,882, which is incorporated herein by reference. Housing 90 defines a patient interface 92. The illustrated patient interface 92 is a mouthpiece. Housing 90 is configured to receive a detachable source 94 of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, via an interface component 96. The interface component 96 communicates with the patient interface 92, and may contain an aerosolization element for aerosolizing the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, before delivery to the patient. The housing 90 may be actuated in a variety of ways, including pressing down on the source 94 of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, such that a predetermined amount is released and travels through the interface component 96 into the patient interface 92.

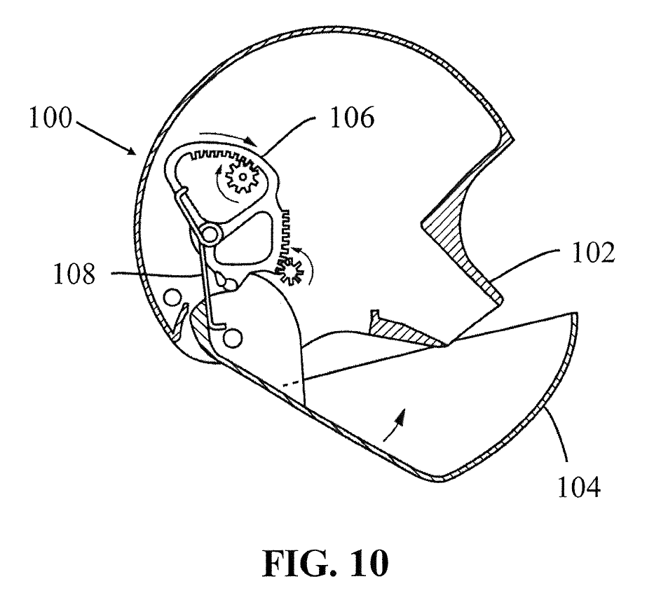

FIG. 10 is a schematic diagram illustrating an alternative embodiment 100 comprising a patient interface mouthpiece 102 and a moveable cover 104, as disclosed in U.S. Pat. No. 5,619,984, which is incorporated herein by reference. Moveable cover 104 may be pivotally mounted on the housing. As a result, when the moveable cover 104 is closed the patient interface 102 is not accessible. Moveable cover 104 is connected to a source delivery mechanism 106 by a linkage 108.

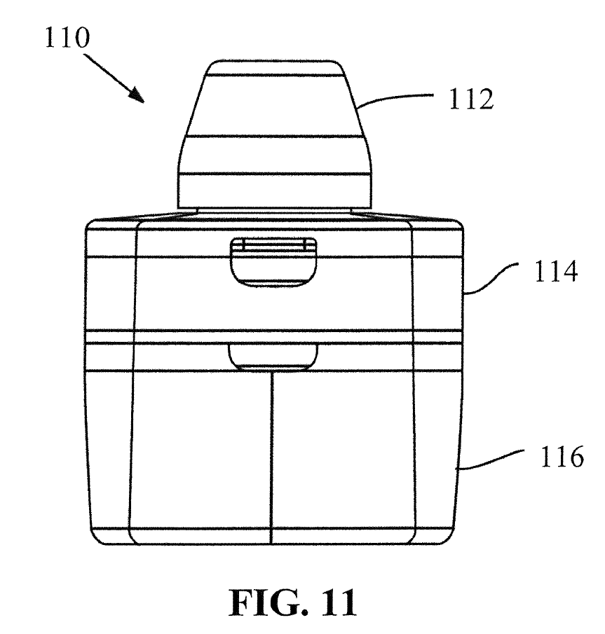

In particular disclosed embodiments, the disclosed xinafoate salt may be contained within a blister pack and administered using, for example, the embodiment of FIG. 11, as disclosed in U.S. Pat. No. 7,318,435, which is incorporated herein by reference. According to FIG. 11, the housing 110 comprises a patient interface mouthpiece 112, a dosage preparation component 114 comprising a flat surface on which the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, to be administered is placed, and a storage base 116 for storing multiple doses of the disclosed xinafoate salt. The storage base 116 may be configured to contain multiple blister packages of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, and may be disassembled from the dosage preparation component 114 and the patient interface 112 in order to retrieve a blister package. The dosage preparation component 114 may also be detached from the patient interface 112 in order to place an appropriate dose of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, in the housing to prepare it for administration to the patient.

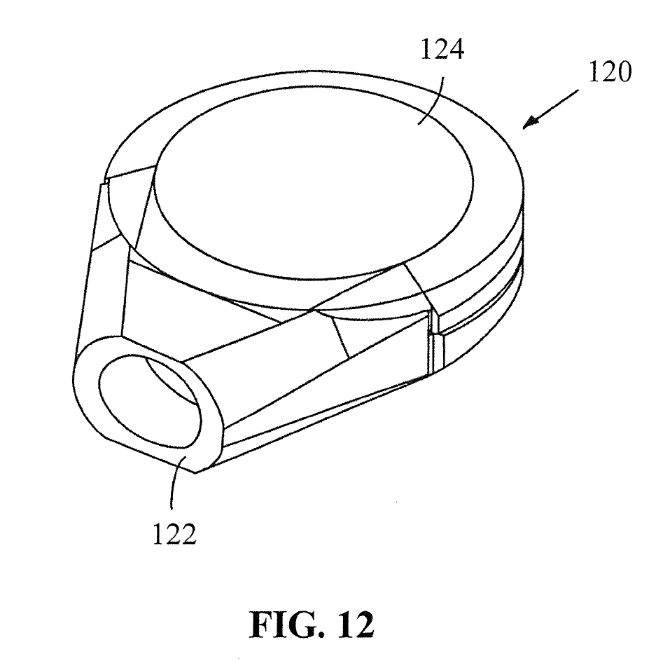

FIG. 12 is a schematic diagram that depicts yet another embodiment 120 as disclosed in U.S. Patent No. 6,116,238, which is incorporated herein by reference. Device 120 comprises a patient interface mouthpiece 122 and a cover 124 that encloses a source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, as a disk of blister packages (not shown). The curvature profile of device 120 allows the device to be held comfortably in a patient's hand. Device 120 also may include sensors and a microprocessor (not shown). When the sensor detects a pressure change induced by the inhalation of a patient, the aerosolization element aerosolizes the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, and delivers it to the patient through the patient interface 122.

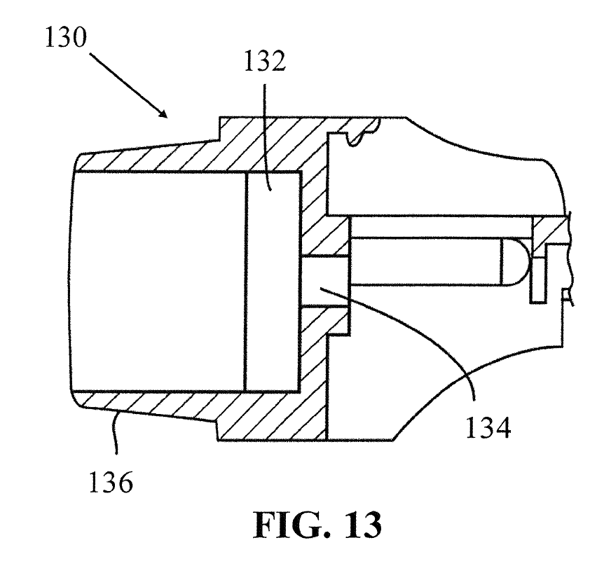

Referring to FIG. 13, device 130 includes an aerosolization chamber 132. Aerosolization chamber 132 communicates with a source of xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof (not shown), via a powder port 134, as disclosed in U.S. Pat. No. 6,116,238. In this embodiment, a predetermined quantity of powdered xinafoate salt enters the aerosolization chamber 132 through the powder port 134 and is aerosolized by gas flow through the aerosolization chamber 132 induced by an impeller (not shown) before passing through the patient interface 136 to the patient.

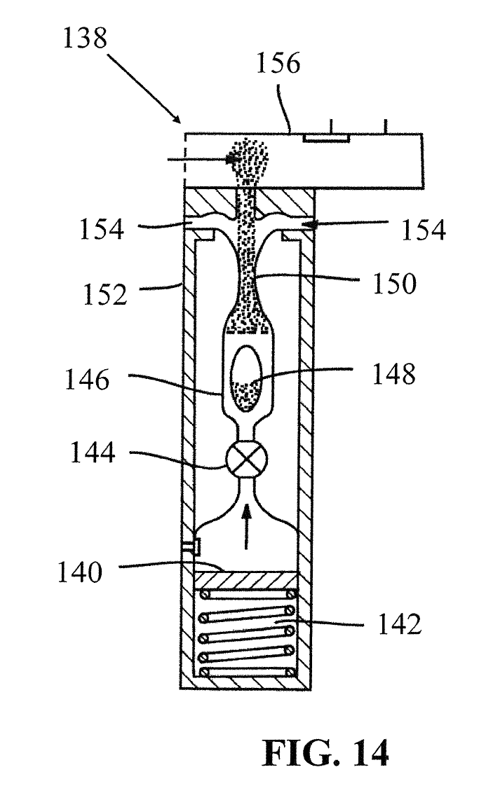

FIG. 14 illustrates device 138 comprising a plunger 140 and spring 142 for compressing a gas that flows through a valve 144 and into an aerosolization chamber 146, as disclosed in U.S. Pat. No. 7,708,011, which is incorporated herein by reference. Aerosolization chamber 146 is configured to contain a predetermined dose of the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. Gas flow into the chamber 146 aerosolizes and deagglomerates the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, 148 by accelerating it through a nozzle 150. The gas then exits the housing 152 through outlets 154. The aerosolized xinafoate salt 148 continues into a patient interface 156 for delivery to a patient. The components of the aerosolization element may be arranged in a linear fashion such that the gas does not change direction during the aerosolization process until it is discharged from the housing.

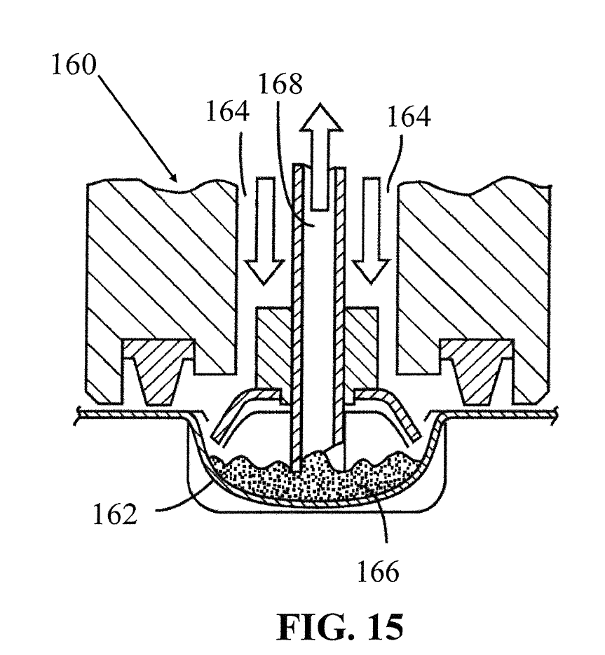

FIG. 15 illustrates a device 160 similar to an embodiment disclosed in U.S. Pat. No. 7,810,494, which is incorporated by reference. With device 160, compressed gas moving in a downward direction from a reservoir (not shown) enters a chamber 162 via conduits 164, aerosolizes the xinafoate salt 166 and carries the aerosolized product up through a central conduit 168 to be delivered to the patient. Chamber 162 both reverses the flow direction of the gas and aerosolizes the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, resulting in an aerosolization element requiring a smaller interior length dimension in a housing. The disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, may be placed in the chamber 162 contained in a blister package. The blister package is pierced by central conduit 168, or may enter the chamber through other channels or conduits in communication with a source of the xinafoate salt. The central conduit 168 also may communicate with an additional aerosolization component, such as a nozzle (not shown), or may connect directly to a patient interface (not shown) depending on the degree of deagglomeration required to achieve a particular particle size.

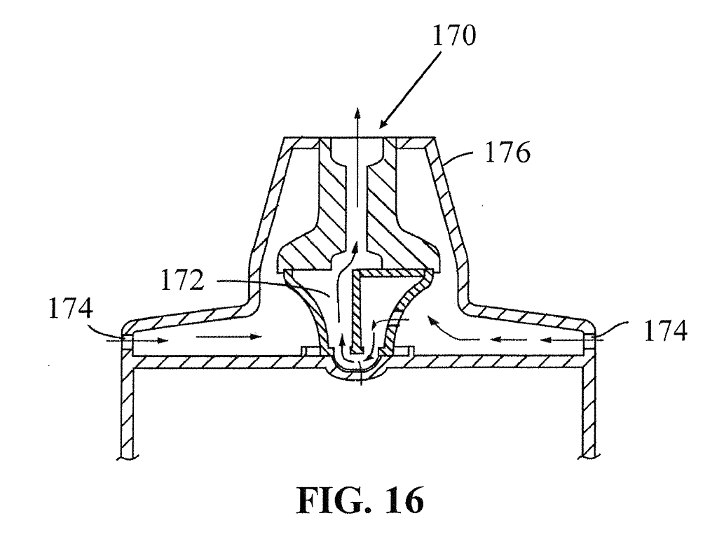

FIG. 16 depicts a device 170 comprising an aerosolization chamber 172. Air is drawn into the chamber 172 through inlets 174. This entrains the xinafoate salt of N4-[(2,2-difluoro-4H-benzo[1,4]oxazin-3 -one)-6-yl]-5-fluoro-N2- [3-(methylaminocarbonylmethyleneoxy)phenyl]-2,4-pyrimidinediamine, or compositions thereof, and carries it up through the patient interface 176, as disclosed in U.S. Pat. No. 7,318,435, which is incorporated herein by reference. The aerosolization element of FIG. 16 operates without the aid of compressed gas or mechanical aerosolizing components. Instead, air flow is induced by patient inhalation, which draws air into the housing through the inlets 174 and into the chamber 172 to entrain and aerosolize the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, through the patient interface 176 for delivery to the patient.

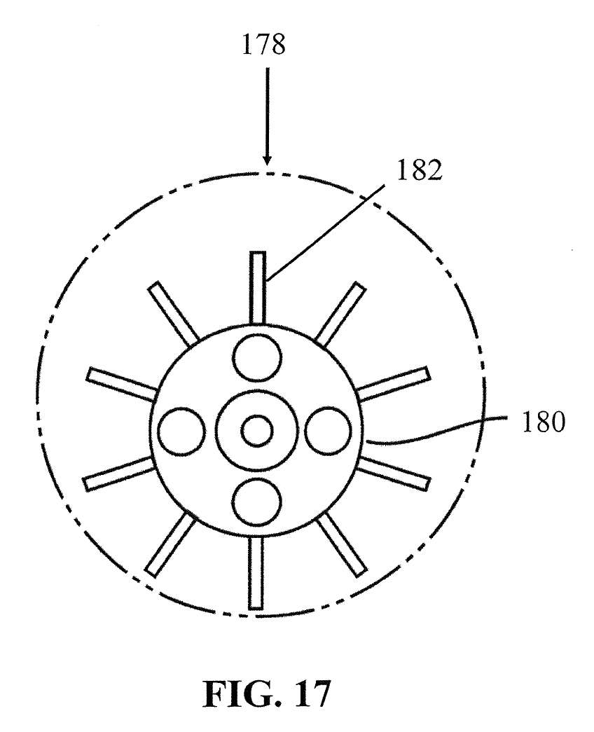

FIG. 17 is a schematic diagram of an embodiment of an impeller 178 having a central hub 180 and a plurality of blades 182 disposed around the central hub. In particular disclosed embodiments, the impeller may be located within an aerosolization chamber or elsewhere in a housing for purposes of aerosolizing a xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. The impeller may be driven by an electric motor, and may induce a flow of gas through a passageway in communication with a patient interface to aerosolize the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, for delivery to a patient.

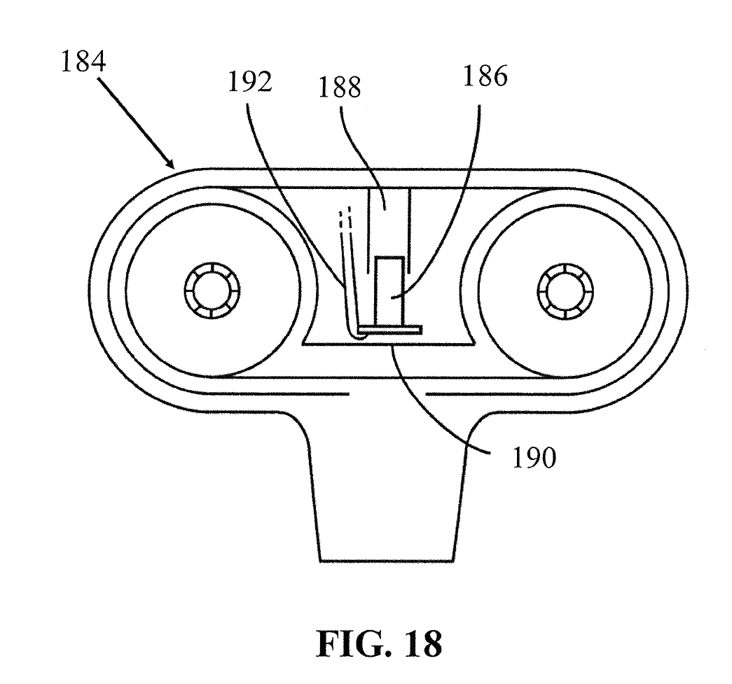

FIG. 18 is a schematic diagram illustrating a device 184 for aerosolizing the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. Device 184 comprises a striking hammer 186 driven by a spring 188, as disclosed in U.S. Pat. No. 5,619,984. The striking hammer 186 and spring 188 are disposed such that the striking hammer strikes a carrier 190 loaded with powdered xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, thereby releasing and aerosolizing the xinafoate salt. The striking hammer 186 and spring 188 are held in the armed position by a catch 192. Releasing catch 192 causes the striking hammer 186 to impact the carrier 190 and aerosolize the powdered xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof, stored by the carrier.

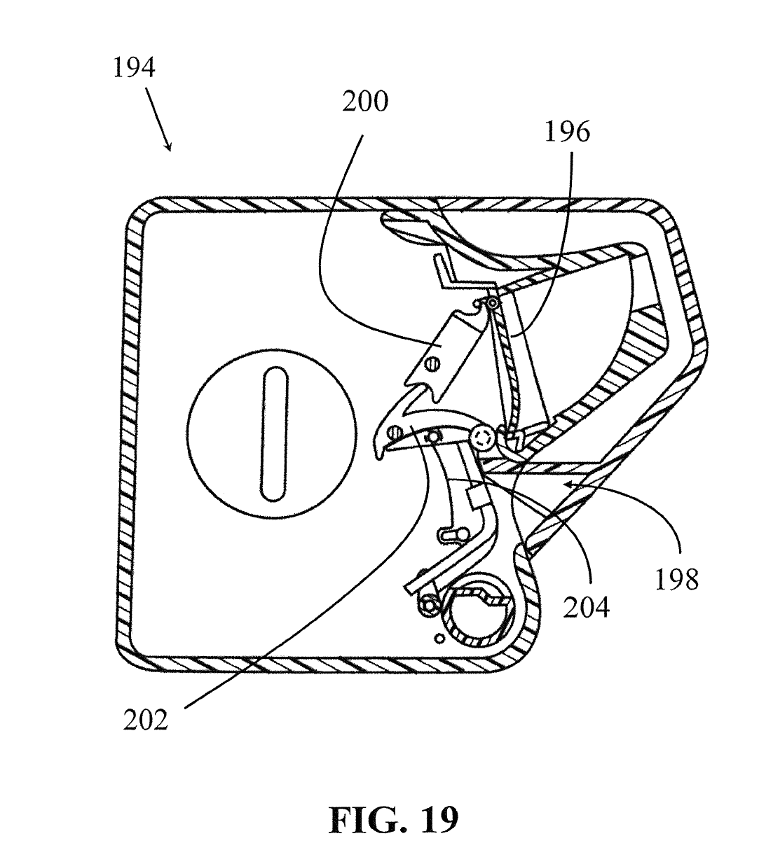

FIG. 19 illustrates a device 194 comprising a triggering mechanism comprising a vane 196 that pivots between closed and open positions, and a reset component 198. Device 194 also comprises a rocker 200, a catch 202, and an activator component 204. FIG. 19 illustrates the device when it is not in use.

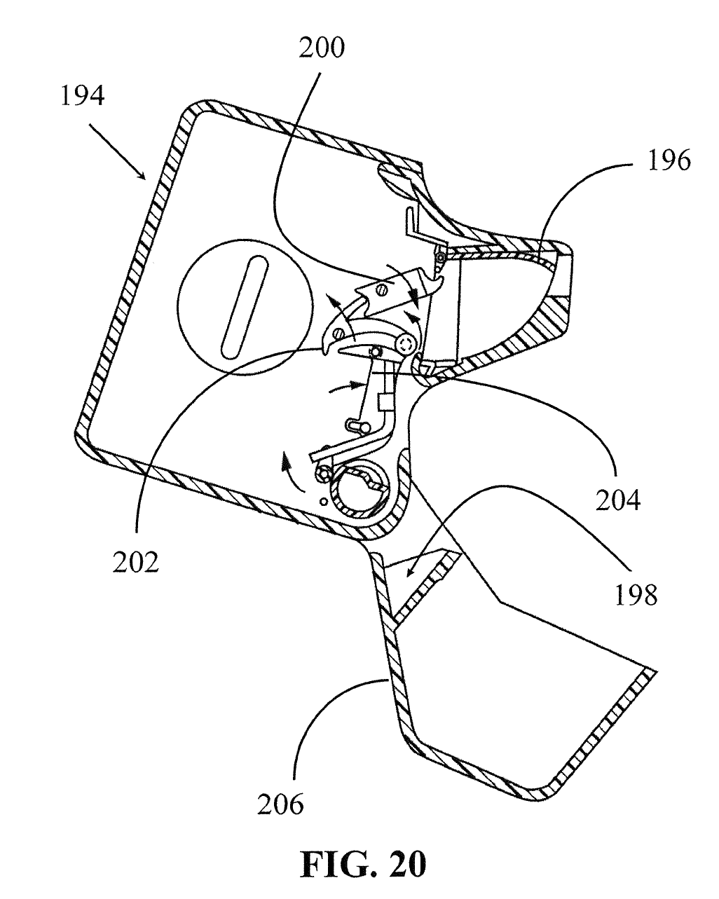

FIG. 20 further illustrates the device 194 when the vane 196 is in its open position due to the interrelated movement between the rocker 200 and the catch 202. According to FIG. 20, the activator component 204 is allowed to move to its dispensing position and the reset component 198 is not in its reset position. In one aspect, movement of vane 196 to its open position provides audible feedback indicating that a dose has been delivered. Similarly, a visual indication of dose delivery may be provided, for example by providing a visual color indicator and/or a dosage counter as discussed below.

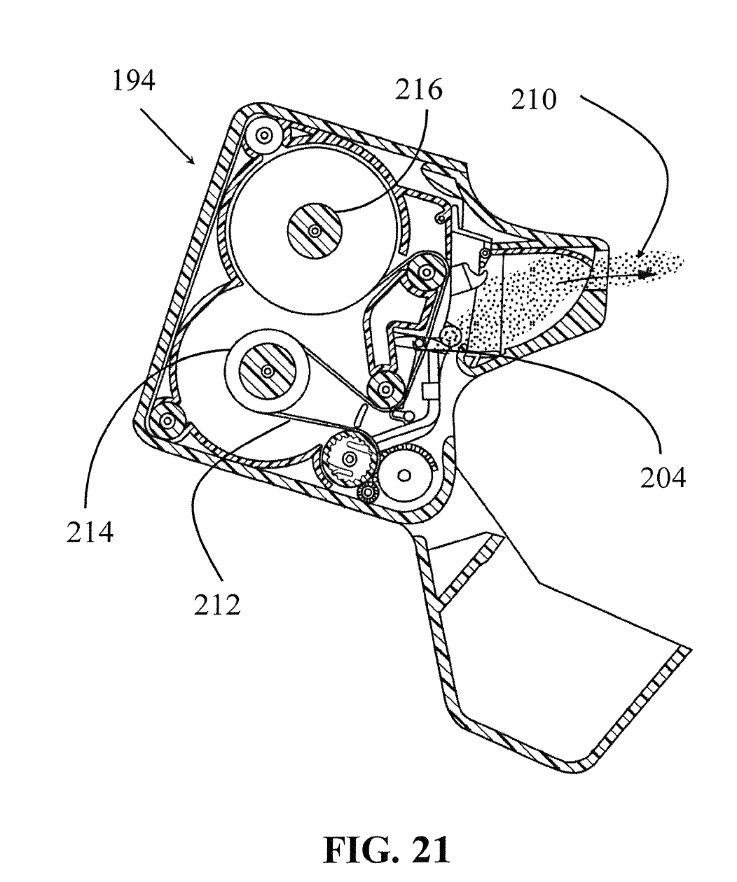

FIG. 21 further illustrates device 194 when in use. According to FIG. 21, device 194 comprises an elongate carrier 212 which comprises the xinafoate salt, or compositions thereof. The device further comprises a cassette, substantially similar to that illustrated in FIG. 7. The activator component 204 works to dispense the xinafoate salt as a free powder 210 by striking the elongate carrier 212. The device may further comprise an advancement mechanism comprising spools 216 and 214, which act to advance the elongate carrier 212 during use.

Embodiments of the device may include a dosage counter. The dosage counter may be provided in the housing. In certain embodiments the dosage counter may include an indicator wheel comprising indicia, such as numbers, for counting dosages administered. See for example, U.S. Pat. No. 7,407,066, which is incorporated herein by reference. In certain embodiments, the device comprises a housing containing a dosage counter comprising at least two annular members and a cog, each mounted rotationally. Actuation of the devices causes a first annular member to incrementally rotate. A predetermined number of actuations causes the cog to rotate, which causes a second annular member to incrementally rotate, allowing dosages to be counted upon rotation. See, for example, U.S. Pat. No. 7,780,038, which is incorporated herein by reference. Incremental rotation of the cog can provide tactile or audible feedback to a user indicating that a dose has been dispensed. For certain embodiments, a dose counter is provided comprising first and second count indicators. The first count indicator has a first indicia bearing surface, and rotates about a first axis, whereas the second count indicator has a second indicia bearing surface, and rotates about a second axis disposed at an obtuse angle with respect to the first axis. The first and second indicia bearing surfaces align at a common viewing area to collectively present a medication dosage count. Alternatively, the first and second axes are not disposed in coaxial, parallel or perpendicular alignments relative to each other, but nevertheless align at a common viewing area to collectively present at least a portion of a medication dosage count. By using first and second counter indicators, a desirable compact counter can be provided which fits into the housing and that has reduced influence on the inhaler airflow. Displaying separate digits or indicia in juxtaposition facilitates reading the counter by a user. Additional information concerning such dosage counters can be found in U.S. Patent Publication No. 2009/0173346, which is incorporated herein by reference. In one embodiment, the device disclosed herein provides audible and/or tactile feedback to indicate that a dose has been delivered, for example, an audible click when a dosage is delivered. Such a device may also include a dosage counter as described. The audible click can occur, for example, when the dose counter advances, or, for example upon actuation of the device.

The dosage counter can be an electrical dosage counter. For these embodiments, the device may include a switch for completing an electrical circuit. A counter module counts dosages when the electrical circuit is opened or closed. The device also can include a display to provide dispensation. Additional information concerning such dosage counters can be found in U.S. Patent Publication No. 2005/0028815, which is incorporated herein by reference.

### V. Method of Making the Disclosed Device

Particular disclosed embodiments concern a method for making a device suitable for administering the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. The device may comprise one or more components that are capable of being operatively associated to form the device. The components contemplated by the present disclosure as are previously recited.

The device may be an inhaler, which comprises an elongate carrier capable of dispensing the disclosed xinafoate salt. The elongate carrier may comprise at least one microdepression. The device may be made by providing an elongate carrier and filling at least one microdepression with the disclosed xinafoate salt. Particular disclosed embodiments concern an elongate carrier that is a microstructured carrier tape. The microstructured carrier tape may comprise at least one microdepression of a size sufficient to hold a dosage of the disclosed xinafoate salt. Typically, at least one microdepression or a plurality of microdepressions together hold a dosage of the disclosed xinafoate salt. Microdepressions suitable for dispensing the present xinafoate salt generally have a depth of from about 5 to about 500 microns and an opening at the surface of the tape that is from about 10 to about 500 microns in width. In certain embodiments depressions are from about 5 to about 150 microns in depth and have an opening at the surface of the tape of from about 50 to about 200 microns in width. Depressions can be spaced at an interval of from about 20 microns to about 1500 microns, such as up to about 2000 microns, for example from about 300 to about 2000 microns apart. The density of depression may be such that there are from about 25 to about 1000 depressions per cm². In one aspect, a microdepression capable of holding at least about 0.05 mg, such as from about 0.1 mg to about 3 mg, such as to about 1 mg or 2mg of the xinafoate salt is used. In another aspect, the xinafoate salt is loaded into the microdepressions such that a single dosage is carried in an area of the elongate carrier that is from about 0.25 to about 2.5 cm², such as from about 1 to about 1.5 or to about 2.25 cm² of the carrier. The microdepressions may be filled with the disclosed xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, or compositions thereof. In one aspect, the xinafoate salt is used without an excipient, for example the microdepression may be filled with neat xinafoate salt. The carrier tape typically is loaded such that from about 25 to about 500 μg, such as from about 50 to about 250 or to about 300 μg of powder, such as neat xinafoate salt are carried per cm². The microdepressions can be filled using an asynchronous roller coating method that uses a coating roller in combination with a feeder to deposit the xinafoate salt, or compositions thereof, into the at least one micro-depression. The coating roller and the elongate carrier may move in the same direction at different linear speeds, and in certain disclosed embodiments, the coating roller has a speed approximately three times faster than the elongate carrier speed. In particular disclosed embodiments, the coating roller is covered with a layer of the xinafoate salt, or compositions thereof, and the feeder has a rate of deposition that matches a rate at which the micro-depression is filled.

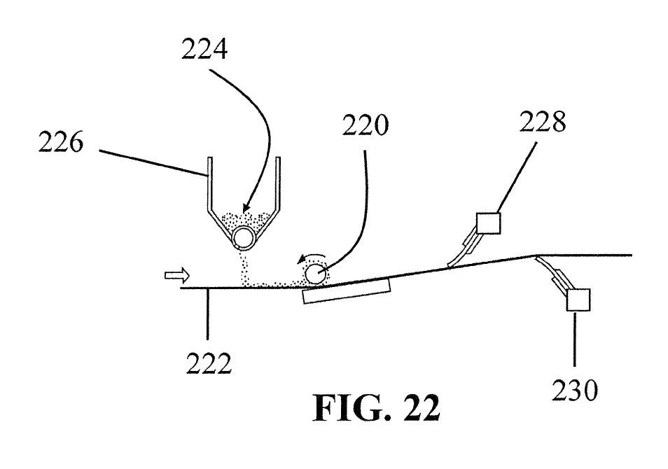

FIG. 22 illustrates an embodiment wherein a coating roller 220, typically covered with the xinafoate salt 224, is used to spread the xinafoate salt, or compositions thereof, over the elongate carrier 222. The xinafoate salt 224, or compositions thereof, may be dispensed from a feeder 226. The elongate carrier may be held flat and stabilized by two blades 228 and 230 as it moves in the same direction as the coating roller 220. Exemplary powder filling processes that can be used to fill microdepressions are described in U.S. Patent Publication No. 2010/0229859.

In particular disclosed embodiments, the device is made by associating the xinafoate salt, or compositions thereof, with the device. In particular disclosed embodiments the xinafoate salt is associated with the device by filling at least one source of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine, compositions thereof, or combinations thereof. In particular disclosed embodiments, associating comprises filling a microdepression and/or introducing an elongate carrier having at least one microdepression filled with the xinafoate salt.

### VI. Method of Using the Disclosed Device

In particular disclosed embodiments, the disclosed device is provided to a patient selected to have a particular malady, such as those disclosed herein, or to a patient who may or may not have a particular malady. Typically, the device is made to fit the patient's hand for patient-initiated use by actuating the device as required for each of the embodiments disclosed herein.

### VII. Xinafoate Salt

The present disclosure relates to a device for administering the xinafoate salt of N4-[(2,2-difluoro-4H-benzo[1,4]oxazin-3-one)-6-yl]-5-fluoro-N2-[3-(methylaminocarbonylmethylene oxy)phenyl]-2,4-pyrimidinediamine and pharmaceutical compositions comprising the disclosed xinafoate salt. Also disclosed is a process for making the xinafoate salt. Certain disclosed embodiments concern using the salt and/or a composition thereof in the treatment of various conditions, particularly in the treatment of inflammatory conditions such as asthma.

The compound N4-[(2,2-difluoro-4H-benzo[1,4]oxazin-3-one)-6-yl]-5-fluoro-N2-[3-(methyl aminocarbonylmethyleneoxy)phenyl]-2,4-pyrimidinediamine, has the following structural formula (I):

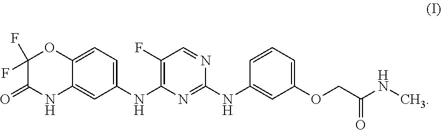 [SMILES: CNC(=O)COc1cccc(Nc2ncc(F)c(Nc3ccc4c(c3)NC(=O)C(F)(F)O4)n2)c1]

N4-[(2,2-difluoro-4H-benzo[1,4]oxazin-3-one)-6-yl]-5-fluoro-N243-(methyl aminocarbonylmethyleneoxy)phenyl]-2,4-pyrimidinediamine is disclosed in WO-A-031063794 as Example 7.3.907 on page 440. The compound, which is also known as 2- {3- [4-(2,2-Difluoro-3-oxo-3,4-dihydro-2H-benzo [1,4]oxazin-6-ylamino)5 -fluoro-pyrimidin-2-ylamino]phenoxy}N-methyl-acetamide, is one of a genus of compounds that are inhibitors of Syk kinase and therefore useful in the treatment of inflammatory conditions, such as chronic obstructive pulmonary disease (COPD). The disclosed compound can be formulated in a pharmaceutical composition in its free form or as a hydrate, solvate, N-oxide, or pharmaceutically acceptable salt. A pharmaceutical composition suitable for inhalation comprising the compound and a suitable powder base, such as lactose or starch, also is disclosed.

The free form of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine disclosed in WO-A-03/063794 is predominantly amorphous, or exists in a disordered crystalline form and is prone to hydration and solvation. Thus, there is a need to provide a new form of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine. Salt formation might provide a form of the compound with better pharmaceutical properties, but the compound may not form salts with many common pharmaceutically acceptable acids. Many salt forms may be obtained, such as the mesylate, fumarate, hemifumarate, hydrobromide, hydrochloride, D-tartrate, hemisulphate and isethionate salts; however, these salts have one or more unsatisfactory properties, such as poor crystallinity and the propensity to form hydrates and/or solvates.

Disclosed herein is an embodiment of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine xinafoic acid salt that has a unique set of characteristics making it suitable for administration as a dry powder formulation. The disclosed xinafoate salt is highly crystalline, has a melting point of about 233° C., is essentially non-hygroscopic, and can be micronized by jet milling without inducing any change in crystalline form. The crystalline form of the disclosed xinafoate salt is thermodynamically stable. It also shows good stability when blended with lactose monohydrate and tested under aggressive conditions of heat and humidity. The lactose blend aerosolizes well when used in conjunction with inhalers, such as, but not limited to, dry powder inhalers.

Disclosed embodiments therefore provide, in a first aspect, the xinafoate salt of N4-[(2,2-difluoro-4H-benzo[1,4]oxazin-3-one)-6-yl]-5-fluoro-N2-[3-(methylaminocarbonyl methyleneoxy)phenyl]-2,4-pyrimidinediamine, having the structure shown in Formula (II) below. A person of ordinary skill in the art will recognize that “xinafoate” and “xinafoic acid” are the common names for 1-hydroxy-2-naphthoate and 1-hydroxy-2-naphthoic acid, respectively. Although Formula II depicts a particular tautomeric form, one of skill in the art will appreciate that the disclosed molecule can be depicted in several different tautomeric forms depending on the location of the proton, all of which are equivalent.

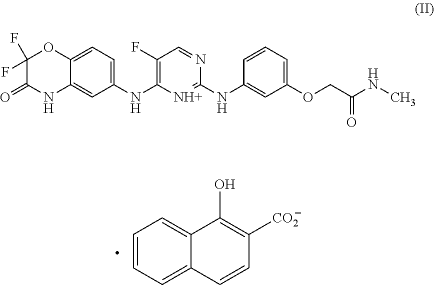

### VIII. Method of Making the Disclosed Xinafoate Salt

The disclosed xinafoate salt can be prepared using any method known to a person of ordinary skill in the art to be suitable for forming a salt derivative of a parent compound. Particular disclosed embodiments concern dissolving N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine and 1-hydroxy-2-naphthoic acid in an organic solvent capable of dissolving the compounds, and allowing a salt, such as a respirable salt, to precipitate. Certain disclosed embodiments concern using between 1 and 1.1 molar equivalents of 1-hydroxy-2-naphthoic acid in a minimum amount of an organic solvent capable of dissolving the parent compound, and cooling the solution slowly, optionally with stirring, until the salt precipitates from the solution. Suitable solvents include, but are not limited to, acetone, acetonitrile and methyl ethyl ketone (MEK), each optionally containing a small amount of water (e.g. less than 10%). Methyl ethyl ketone is particularly suitable and is preferably used with about 5% by volume of water. The reactants are typically dissolved in the solvent at a temperature higher than room temperature but below the boiling point of the solvent.

N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine may be prepared by the general and specific methods disclosed in WO-A-03/063794. It may, for example, be prepared by reacting a compound of Formula III

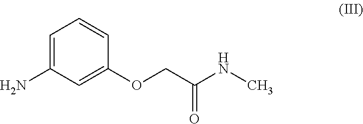

with a compound of Formula IV

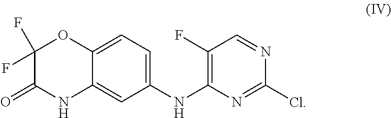 [SMILES: O=C1Nc2cc(Nc3nc(Cl)ncc3F)ccc2OC1(F)F]

The reaction is typically carried out in a suitable solvent, preferably an alcohol, such as isoamyl alcohol or isopropyl alcohol, and in the presence of an acid catalyst, such as trifluoroacetic acid. The reaction is best carried out at an elevated temperature. For example, if amyl alcohol is selected as the solvent, a temperature of about 100° C. is preferred.

A compound of formula (III) may be prepared by the route set out in Scheme 1 below.

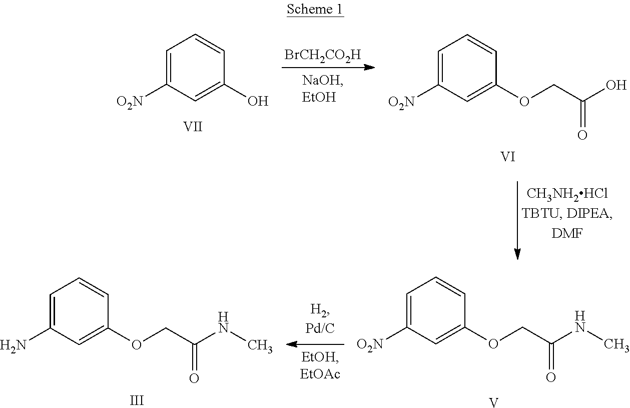

A compound of formula (III) may be prepared by reducing the nitro group in a compound of formula (V). In a preferred procedure, hydrogenation is used. Typically, a solution of the compound of formula (V) in a suitable organic solvent, such as a mixture of ethanol (EtOH) and ethyl acetate (EtOAc), is treated with a hydrogenation catalyst, such as palladium on carbon, and exposed to hydrogen gas. The hydrogen is usually applied at a pressure above atmospheric, such as at 30 pounds per square inch (psi).

A compound of formula (V) may be prepared by condensing the acid of formula (VI) with methylamine, or a salt thereof (such as the hydrochloride salt). Any condensing agent suitable for the formation of amide bonds may be used in principle, but the use of 2-(1H-benzatriazole-1-yl)-1,1,3,3-tetramethyluronium tetrafludroborate (TBTU) is preferred. The condensation catalyzed by TBTU is carried out in a suitable organic solvent, such as N,N-dimethylformamide (DMF), and in the presence of a base such as N,N-diisopropylethylamine (DIPEA).

A compound of foiinula (VI) may be prepared by alkylating 3-nitrophenol (VII) with bromoacetic acid. The reaction is typically carried out in a suitable solvent, such as water or aqueous ethanol (EtOH), in the presence of a base, such as sodium hydroxide (NaOH), and at elevated temperature, e.g. at the reflux temperature of the chosen solvent.

A compound of formula (IV) can be prepared by the route set out in Scheme 2 below.

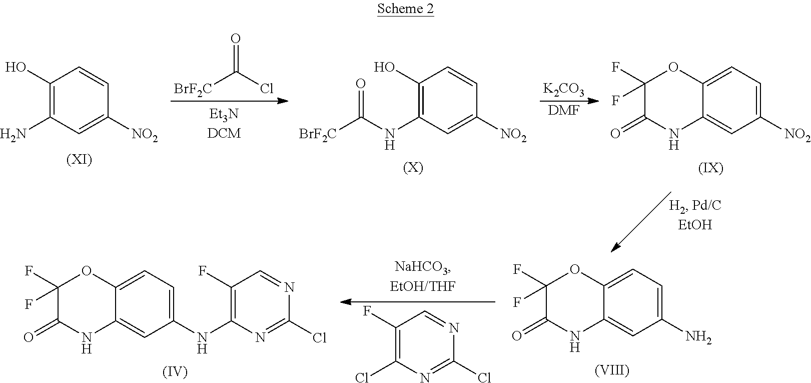

A compound of formula (IV) may be prepared by reacting a compound of formula (VIII) with 5-fluoro-2,4-dichloropyrimidine. In a typical procedure, a solution of the reactants in a suitable organic solvent, such as ethanol (EtOH) or a mixture of ethanol and tetrahydrofuran (THF), is treated with a base such as sodium hydrogencarbonate.

A compound of formula (VIII) may be prepared by reducing the nitro group in a compound of formula (IX). In a preferred procedure, hydrogenation is used. Typically, a solution of the compound of formula (IX) in a suitable organic solvent, such as ethanol (EtOH), is treated with a hydrogenation catalyst, such as palladium on carbon, and exposed to hydrogen gas. The hydrogen is usually applied at a pressure above atmospheric, such as at 30 pounds per square inch (psi).

A compound of formula (IX) may be prepared by cyclization of a compound of formula (X). In a typical procedure, a solution of a compound of formula (X) in a suitable organic solvent, such as N,N-dimethylformamide (DMF) or isopropyl acetate, is treated with a base, such as potassium carbonate, and heated, for example at the reflux temperature of the solvent. When DMF is chosen as the solvent, a temperature of about 120° C. may be used. When isopropyl acetate is chosen as solvent, a temperature of about 85° C. may be used.

A compound of formula (X) may be prepared by acylation of the aniline of formula (XI) with 2-bromo-2,2-difluoroacetyl chloride. The reaction is preferably carried out in a suitable organic solvent, such as dichloromethane (DCM) or acetonitrile, in the presence of a base, such as triethylamine. The reaction is exothermic and cooling, for example to 0° C., may therefore be required.

Disclosed embodiments include all crystalline and pharmaceutically acceptable isotopically-labeled forms of the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine. In an isotopically-labeled form, one or more atoms are replaced by an atom or atoms having the same atomic number, but an atomic mass or mass number different from the atomic mass or mass number which predominates in nature. A person of ordinary skill in the art will recognize that any atom at any position of the disclosed compounds may be isotopically enriched, labeled with at least one isotope, or combinations thereof, with any isotope currently known or discovered in the future. Particular examples of isotopes include isotopes of carbon, hydrogen, nitrogen, oxygen, phosphorous, halogens (e.g. chlorine, fluorine, bromine, and iodine), and combinations thereof.

Suitable isotopes include isotopes of hydrogen, such as ²H and ³H; carbon, such as “C, ¹³C and ¹⁴C; nitrogen, such as ¹³N and ¹⁵N; oxygen, such as ¹⁵0, ¹⁷0 and ¹⁸0; and sulphur, such as ³⁵S. Certain isotopically-labeled compounds, such as those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, for example ²H, may afford certain therapeutic advantages resulting from greater metabolic stability, for example, increased in vivo half-life or reduced dosage requirements, and hence may be preferred in some circumstances. Accordingly, a compound may be enriched, relative to natural abundance, in deuterium at one or more positions to provide improved properties. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described herein using an appropriate isotopically-labeled reagent in place of the non-labeled reagent previously employed.

### IX. Method of Using the Disclosed Xinafoate Salt

N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine is a Syk kinase inhibitor and is able to inhibit the degranulation of immune cells, such as mast, basophile, neutrophil and/or eosinophil cells. It may be useful, in the form of the xinafoate salt disclosed herein and otherwise, in the treatment of the following conditions:
Respiratory disorders, including treatable obstructive, restrictive or inflammatory airways diseases of whatever type, etiology, or pathogenesis, in particular an obstructive, restrictive or inflammatory airways disease such as:
   asthma, in particular atopic asthma, allergic asthma, atopic bronchial IgE-mediated asthma, non-atopic asthma, bronchial asthma, non-allergic asthma, essential asthma, true asthma, intrinsic asthma caused by pathophysiologic disturbances, essential asthma of unknown or unapparent cause, emphysematous asthma, exercise-induced asthma, emotion-induced asthma, extrinsic asthma caused by environmental factors, cold air induced asthma, occupational asthma, infective asthma caused by or associated with bacterial, fungal, protozoal, or viral infection, incipient asthma, wheezy infant syndrome, bronchiolitis, cough variant asthma or drug-induced asthma;
   bronchial hyper-responsivity to environmental agents;
   rhinitis or sinusitis of whatever type, etiology, or pathogenesis, in particular seasonal allergic rhinitis, perennial allergic rhinitis, perennial rhinitis, vasomotor rhinitis, post-nasal drip, purulent or nonpurulent sinusitis, acute or chronic sinusitis and ethmoid, frontal, maxillary, or sphenoid sinusitis;
   chronic obstructive pulmonary disease (COPD), chronic obstructive lung disease (COLD), chronic obstructive airways disease (COAD) or small airways obstruction of whatever type, etiology, or pathogenesis, in particular chronic bronchitis, pulmonary emphysema, bronchiectasis, cystic fibrosis, bronchiolitis obliterans, bronchiolitis obliterans organizing pneumonia (BOOP), chronic organizing pneumonia (COP), bronchiolitis fibrosa obliterans, follicular bronchiolitis or dyspnea associated therewith;
   bronchitis of whatever type, etiology, or pathogenesis, in particular acute bronchitis, acute laryngotracheal bronchitis, arachidic bronchitis, catarrhal bronchitis, croupus bronchitis, chronic bronchitis, dry bronchitis, infectious asthmatic bronchitis, productive bronchitis, staphylococcus or streptococcal bronchitis and vesicular bronchitis;
   bronchiectasis of whatever type, etiology, or pathogenesis, in particular cylindric bronchiectasis, sacculated bronchiectasis, fusiform bronchiectasis, capillary bronchiectasis, cystic bronchiectasis, cystic fibrosis, Kartageners's syndrome, dry bronchiectasis or follicular bronchiectasis;
   pulmonary eosinophilic syndromes of whatever type, etiology, or pathogenesis, in particular acute eosinophilic pneumonia (idiopathic or due to drugs or parasites), simple pulmonary eosinophilia, Loeffler's syndrome, tropical pulmonary eosinophilia, chronic eosinophilic pneumonia, allergic bronchopulmonary mycosis, allergic bronchopulmonary aspergillosis (ABPA), Churg-Strauss syndrome or idiopathic hypereosinophilic syndrome;
   interstitial lung diseases (ILD) or pulmonary fibrosis of whatever type, etiology, or pathogenesis, in particular idiopathic pulmonary fibrosis, crytogenic fibrosing alveolitis, fibrosing alveolitis, ILD or pulmonary fibrosis associated with connective tissue disease (systemic lupus erythematosis, mixed connective tissue disease, polymyositis, dermatomyositis, Sjórgen's syndrome, systemic sclerosis, scleroderma, rheumatoid arthritis), usual interstitial pneumonia (UIP), desquamative interstitial pneumonia (DIP), granulomatous lung disease, sarcoidosis,

Wegener's granulomatosis, histiocytosis X, Langerhan's cell granulomatosis, hypersensitivity pneumonitis, extrinsic allergic alveolitis, silicosis, chronic eosinophilic pneumonia, lymphangiolyomatosis, drug-induced ILD or pulmonary fibrosis, radiation-induced ILD or pulmonary fibrosis, alveolar proteinosis, graft-versus-host-disease (GVHD), lung transplant rejection, ILD or pulmonary fibrosis due to environmental/occupational exposure, BOOP, COP, bronchiolitis fibrosa obliterans, follicular bronchiolitis, idiopathic acute interstitial pneumonitis (Hamman Rich syndrome) or alveolar hemorrhage syndromes;
pneumoconiosis of whatever type, etiology, or pathogenesis, in particular aluminosis or bauxite workers' disease, anthracosis or miners' asthma, progressive massive fibrosis (PMF), asbestosis or steam-fitters' asthma, chalicosis or flint disease, ptilosis caused by inhaling the dust from ostrich feathers, siderosis caused by the inhalation of iron particles, silicosis or grinders' disease, byssinosis or cotton-dust asthma or talc pneumoconiosis;
Acute Respiratory Distress Syndrome (ARDS), adult respiratory distress syndrome or acute lung injury of whatever type, etiology, or pathogenesis;
aspiration disorders of whatever type, etiology, or pathogenesis leading to aspiration pneumonitis or aspiration pneumonia;
alveolar hemorrhage of whatever type, etiology, or pathogenesis, in particular a member of the group consisting of idiopathic pulmonary hemosiderosis, alveolar hemorrhage due to drugs or other exogenous agents, alveolar hemorrhage associated with HIV or bone marrow transplant or autoimmune alveolar hemorrhage (e.g. associated with systemic lupus erythematosis, Goodpasture's syndrome, Wegener's granulomatosis, microscopic polyangiitis, Churg-Strauss syndrome, pauci-immune glomerulonephritis);
acute or chronic laryngitis or pharyngitis;
cough of whatever type, etiology, or pathogenesis in particular idiopathic cough or cough associated with gastro-esophageal reflux disease (GERD), drugs, bronchial hyper-responsivity, asthma, COPD, COLD, COAD, bronchitis, bronchiectasis, pulmonary eosinophilic syndromes, pneumoconiosis, interstitial lung disease, pulmonary fibrosis, aspiration disorders, rhinitis, laryngitis or pharyngitis;
anaphylaxis and type 1 hypersensitivity reactions of whatever aetiology;
atopic, allergic, autoimmune or inflammatory skin disorders of whatever type, etiology, or pathogenesis, in particular atopic dermatitis, allergic dermatitis, contact dermatitis, allergic or atopic eczema, lichen planus, mastocytosis, erythema nodosum, erythema multiforme, benign familial pemphigus, pemphigus erythematosus, pemphigus foliaceus, and pemphigus vulgaris, bullous pemphigoid, epidermolysis bullosa, dermatitis hepetiformis, psoriasis, immune-mediated urticaria, complement-mediated urticaria, urticariogenic material-induced urticaria, physical agent-induced urticaria, stress-induced urticaria, idiopathic urticaria, acute urticaria, chronic urticaria, angioedema, cholinergic urticaria, cold urticaria in the autosomal dominant form or in the acquired form, contact urticaria, giant urticaria or papular urticaria;
conjunctivitis of whatever type, etiology, or pathogenesis, in particular actinic conjunctivitis, acute catarrhal conjunctivitis, acute contagious conjunctivitis, allergic conjunctivitis, atopic conjunctivitis, chronic catarrhal conjunctivitis, purulent conjunctivitis or vernal conjunctivitis;
multiple sclerosis of whatever type, etiology, or pathogenesis, in particular primary progressive multiple sclerosis or relapsing remitting multiple sclerosis;
autoimmune/inflammatory diseases of whatever type, etiology, or pathogenesis, in particular autoimmune hematological disorders, hemolytic anemia, aplastic anemia, pure red cell anemia, idiopathic thrombocytopenic purpura, rheumatoid arthritis, systemic lupus erythematosus, scleroderma, systemic sclerosis, oolymyalgia rheumatica, dermatomyositis, polymyositis, polychondritis, Wegner's granulomatosis, chronic active hepatitis, myasthenia gravis, Stevens-Johnson Johnson syndrome, idiopathic sprue, autoimmune inflammatory bowel diseases, Crohn's disease, ulcerative colitis, endocrine opthalmopathy, Grave's disease, sarcoidosis, alveolitis, chronic hypersensitivity pneumonitis, primary biliary cirrhosis, juvenile diabetes or diabetes mellitus type I₁ keratoconjunctivitis sicca, epidemic keratoconjunctivitis, glomerulonephritis with or without nephrotic syndrome, acute glomerulonephritis, idiopathic nephrotic syndrome, minimal change nephropathy, autoimmune disorders associated with interstitial lung disease and/or pulmonary fibrosis or autoimmune or inflammatory skin disorders;
inflammatory bowel disease (IBD) of whatever type, etiology, or pathogenesis, in particular collagenous colitis, colitis polyposa, transmural colitis, ulcerative colitis or Crohn's disease (CD);
pulmonary hypertension of whatever type, etiology or pathogenesis including pulmonary arterial hypertension, pulmonary venous hypertension, pulmonary hypertension associated with disorders of the respiratory system and/or hypoxemia, pulmonary hypertension due to chronic thrombotic and/or embolic disease and pulmonary hypertension due to disorders directly affecting the pulmonary vasculature;
arthritis of whatever type, etiology, or pathogenesis, in particular rheumatoid arthritis, osteoarthritis, gouty arthritis, pyrophosphate arthropathy, acute calcific periarthritis, chronic inflammatory arthritis, arthritis associated with a connective tissue disorder (e.g. systemic lupus erythematosis, polymyositis, dermatomyositis, systemic sclerosis, scleroderma), sarcoidosis, polymyalgia rheumatica, degenerative arthritis, infectious arthritis, Lyme arthritis, proliferative arthritis, psoriatic arthritis, ankylosing spondylitis, cervical spondylosis, vertebral arthritis, juvenile arthritis (Still's disease), amyloidosis, ankylosing vertebral hyperostosis (Forrestier's disease), Behcet's syndrome, drug-induced arthritis, familial Mediterranean fever, hypermobility syndrome, osteochondritis dessicans, osteochondromatosis, palindromic rheumatism, pigmented villonodular synovitis, relapsing polychondritis, temporomandibular pain dysfunction syndrome or arthritis associated with hyperlipidemia;
an eosinophil-related disorder of whatever type, etiology, or pathogenesis, in particular pulmonary eosinophilic syndromes, aspergilloma, granulomas containing eosinophils, allergic granulomatous angiitis or Churg-Strauss syndrome, polyarteritis nodosa (PAN) or systemic necrotizing vasculitis;
uveitis of whatever type, etiology, or pathogenesis, in particular inflammation of all or part of the uvea, anterior uveitis, iritis, cyclitis, iridocyclitis, granulomatous uveitis, nongranulomatous uveitis, phacoantigenic uveitis, posterior uveitis, choroiditis or chorioretinitis;
septic shock of whatever type, etiology, or pathogenesis;
disorders of bone deposition/resorption, including osteoporosis and osteopenia;
lymphoproliferative disorders (e.g. lymphoma, myeloma);
HIV or AIDs related disorders;
infection, especially infection due to viruses wherein such viruses increase the production of TNF-α in their host, or wherein such viruses are sensitive to upregulation of TNF-α in their host so that their replication or other vital activities are adversely impacted, including a virus which is a member selected from the group consisting of HIV-1, HIV-2, and HIV-3, cytomegalovirus (CMV), influenza, adenoviruses and Herpes viruses including Herpes zoster and Herpes simplex;
yeast and fungal infections wherein the yeast or fungus is sensitive to upregulation by TNF-α or elicits TNF-α production in the host, e.g., fungal meningitis, particularly when administered in conjunction with other drugs of choice for the treatment of systemic yeast and fungus infections, including but are not limited to, polymixins (e.g. Polymycin B), imidazoles (e.g. clotrimazole, econazole, miconazole, and ketoconazole), triazoles (e.g. fluconazole and itranazole) and amphotericins (e.g. Amphotericin B and liposomal Amphotericin B); and
Mycobacterial infections e.g. due to mycobacterium tuberculosis.

### X. Compositions Comprising the Disclosed Xinafoate Salt

The xinafoate salt of N4-[(2,2-difluoro-4H-benzo[1,4]oxazin-3-one)-6-yl]-5-fluoro-N2-[3-(methylaminocarbonylmethyleneoxy) phenyl]-2,4-pyrimidinediamine may be administered alone or as a formulation in association with one or more pharmaceutically acceptable excipients. The term “excipient” is used herein to describe any ingredient other than the disclosed xinafoate salt. The choice of excipient will to a large extent depend on factors such as the particular mode of administration, the effect of the excipient on solubility and stability, and the nature of the dosage form. By way of example, lactose is one excipient useful for formulating the disclosed xinafoate salt for inhalation. The term “excipient-free” as used herein refers to a formulation that does not include an excipient, i.e. , the formulation includes only the xinafoate salt.

Pharmaceutical compositions suitable for the delivery of the disclosed xinafoate salt and methods for their preparation will be known to a person of ordinary skill in the art. Such compositions and methods for their preparation may be found, for example, in Remington's Pharmaceutical Sciences, 19th Edition (Mack Publishing Company, 1995), which is incorporated herein by reference.

The disclosed xinafoate salt can also be administered intranasally or by inhalation, typically in the form of a dry powder (either alone, as a mixture, for example, in a dry blend with lactose, or as a mixed component particle, for example, mixed with phospholipids, such as phosphatidylcholine) from an inhaler, such as a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. Administration in the form of a dry powder from a dry powder inhaler is a particularly preferred form of delivery.

In particular disclosed embodiments, the disclosed xinafoate salt is administered using an inhaler capable of administering a respirable dose or a fine particle dose. In particular disclosed embodiments, the inhaler is suitable for administering the highest possible respirable or fine particle dose. In certain disclosed embodiments, the inhaler may deliver a fine particle dose of at least 15%. The inhaler may deliver from about 65% to about 135% of a label claim dose. Particular disclosed embodiments concern delivering about 75% to about 125% of the label claim dose.

The pressurized container, pump, spray, atomizer or nebulizer contains a solution or suspension of the disclosed xinafoate salt comprising, for example, ethanol, aqueous ethanol or a suitable alternative agent for dispersing, solubilizing or extending release of the active, a propellant(s) as solvent and an optional surfactant, such as sorbitan trioleate, oleic acid or an oligolactic acid.

Prior to use in a dry powder or suspension formulation, the drug product is micronized to a size suitable for delivery by inhalation. In particular disclosed embodiments, the disclosed xinafoate salt may have a mean particle size suitable for administration. Typically, the respirable dose of the xinafoate salt will comprise particles of the xinafoate salt having a mean particle size ranging from greater than zero to about 10 μm; preferably from about 0.4 μm to about 5 μm, or about 0.5 μm to about 5 μm. This may be achieved by any appropriate comminuting method, such as spiral jet milling, fluid bed jet milling, supercritical fluid processing, high pressure homogenization or spray drying. In one method for producing a micronized formulation of drug product, the drug product composition is subjected to wet milling.

Wet milling typically produces a distinct formulation as compared to jet milling. Wet milled formulations of the present xinafoate salt may have a narrower particle size distribution and include particles with smoother surfaces, both factors that contribute to increased delivery efficiency.

In some embodiments a dry powder of the disclosed xinafoate salt is encapsulated. Capsules (made, for example, from gelatin or hydroxypropylmethylcellulose), blisters and cartridges for use in an inhaler or insufflator may be formulated to contain a powder mix of the disclosed xinafoate salt, a suitable powder base such as lactose or starch and a performance modifier such as L-leucine, mannitol or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1μg to 20 mg of the disclosed xinafoate salt per actuation and the actuation volume may vary from 1 μl to 100 μl. A typical formulation may comprise a compound of formula II, propylene glycol, sterile water, ethanol and sodium chloride. Alternative solvents which may be used instead of propylene glycol include glycerol and polyethylene glycol.

Suitable flavoring agents, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release includes delayed, sustained, pulsed, controlled, targeted and programmed release.

In the case of dry powder inhalers and aerosols, the dosage unit may be determined by a valve which delivers a metered amount. The overall daily dose may be administered in a single dose or, more usually, as divided doses throughout the day.

For administration to human patients, the total daily dose of the disclosed xinafoate salt of N4-[(2,2-difluoro-4H-benzo[1,4]oxazin-3-one)-6-yl]-5-fluoro-N2-[3-(methyl aminocarbonylmethyleneoxy)phenyl]-2,4-pyrimidinediamine will typically be in the range from about 0.001 mg/kg to about 200 mg/kg depending, of course, on the mode of administration. Typical dosages of the disclosed xinafoate salt for inhalation range from about 0.005 to about 20 mg/kg, such as from about 0.01 to about 10 mg/kg, such as from about 0.01 to about 1 mg/kg, or from about 0.04 to about 0.8 mg/kg, or from about 0.05 to about 2 mg/kg. In some examples, the dosage is at least about 0.01 mg/kg, at least about 0.02 mg/kg, at least about 0.03 mg/kg, at least about 0.04 mg/kg, at least about 0.05 mg/kg, at least about 0.06 mg/kg, at least about 0.07 mg/kg, at least about 0.08 mg/kg, at least about 0.09 mg/kg, at least about 0.1 mg/kg, at least about 0.15 mg/kg, at least about 0.3 mg/kg, at least about 0.5 mg/kg, or at least about 1 mg/kg. The total daily dose may be administered in single or divided doses and may, at the physician's discretion, fall outside of the typical range given herein. Particular disclosed embodiments concern a dosage amount ranging from about 0.5 mg to about 10 mg of the xinafoate salt once or twice per day, such as from about 1 mg to about 4 mg. One exemplary dosage regimen uses 1 mg of xinafoate salt twice daily and a second regimen uses 3 mg twice daily. The respirable dose (e.g. a fine particle dose) delivered with an inhaler ranges from about 10% to about 90% of the theoretical or fill weight of xinafoate salt. According to convention, the term “dose” as used herein refers to the theoretical dose, unless otherwise indicated by context. Disclosed inhaler devices typically are more efficient, delivering a fine particle dose of at least about 15%, such as from about 20% to about 90%, from about 30% to about 80%, from about 50% to about 90%, or at least about 75% of the fill weight.

For the avoidance of doubt, references herein to “treatment” include references to curative, palliative and prophylactic treatment.

Syk kinase inhibitors, such as the disclosed xinafoate salt, may advantageously be administered in combination with one or more other therapeutic agents, particularly in the treatment of respiratory diseases such as asthma. The disclosed xinafoate may be used in combination with one or more other therapeutic agents to make a composition comprising greater than about 0 percent to less than about 100 percent of the disclosed xinafoate salt. Particular disclosed embodiments concern a composition comprising about 1% to about 99%, from about 1% to about 90%, from about 1% to about 80%, from about 1% to about 70%, from about 1% to about 60%, from about 1% to about 50%, from about 1% to about 40%, from about 1% to about 30%, from about 1% to about 20%, from about 1% to about 10%, and from about 1% to about 5% of the disclosed xinafoate salt. Particular disclosed embodiments concern a composition comprising from about 1% to about 20% of the disclosed xinafoate salt and from about 99% to about 80% of the pharmaceutically acceptable carrier, such as lactose.

Examples of such further therapeutic agents include: (i) 5-lipoxygenase (5-LO) inhibitors or 5-lipoxygenase activating protein (FLAP) antagonists; (ii) leukotriene antagonists (LTRAs) including antagonists of LTB₄, LTC₄, LTD₄, and LTE₄; (iii) histamine receptor antagonists including H₁, H₃ and H₄ antagonists; (iv) Oc₁-and α₂-adrenoceptor agonist vasoconstrictor sympathomimetic agents for nasal decongestant use; (v) muscarinic M₃ receptor antagonists or anticholinergic agents; (vi) PDE inhibitors, e.g. PDE₃, PDE₄ and PDE₅ inhibitors; (vii) theophylline; (viii) sodium cromoglycate; (ix) COX inhibitors both non-selective and selective COX-1 or COX-2 inhibitors (NSAIDs); (x) oral and inhaled glucocorticosteroids, such as DAGR (dissociated agonists of the corticoid receptor); (xi) monoclonal antibodies active against endogenous inflammatory entities; (xii) anti-tumor necrosis factor (anti-TNF-α) agents; (xiii) adhesion molecule inhibitors including VLA-4 antagonists; (xiv) WnJn-B₁- and B₂-receptor antagonists; (xv) immunosuppressive agents; (xvi) inhibitors of matrix metalloproteases (MMPs); (xvii) tachykinin NK₁, NK₂ and NK₃ receptor antagonists; (xviii) elastase inhibitors; (xix) adenosine A₂₃ receptor agonists; (xx) inhibitors of urokinase; (xxi) compounds that act on dopamine receptors, e.g. D₂ agonists; (xxii) modulators of the NFKP pathway, e.g. IKK inhibitors; (xxiii) modulators of cytokine signaling pathways such as a p38 MAP kinase or JAK kinase inhibitor; (xxiv) agents that can be classed as mucolytics or anti-tussive; (xxv) antibiotics; (xxvi) HDAC inhibitors; (xxvii) PI3 kinase inhibitors; (xxviii) 13₂ agonists; and (xxix) dual compounds active as β₂ agonists and muscarinic M₃ receptor antagonists. Preferred examples of such therapeutic agents include: (a) glucocorticosteroids, in particular inhaled glucocorticosteroids with reduced systemic side effects, flunisolide, triamcinolone acetonide, beclomethasone dipropionate, budesonide, fluticasone propionate, ciclesonide, and mometasone furoate; (b) muscarinic M₃ receptor antagonists or anticholinergic agents including ipratropium salts such as the bromide, tiotropium salts such as the bromide, oxitropium salts such as the bromide, perenzepine and telenzepine; and (c) β₂ agonists including salbutamol, terbutaline, bambuterol, fenoterol, salmeterol, formoterol, tulobuterol. Any of the agents specifically mentioned may optionally be used in the form of a pharmaceutically acceptable salt.

### XI. Kits Comprising the Disclosed Xinafoate Salt

Where it is desirable to administer a combination of active compounds, two or more pharmaceutical compositions, at least one of which contains the disclosed xinafoate salt, conveniently may be combined in a kit. A kit may comprise two or more separate pharmaceutical compositions, at least one of which contains the disclosed xinafoate salt, and components for separately retaining said compositions, such as a container, divided bottle, or divided foil packet. An example of such a kit is the familiar blister pack used for the packaging of tablets, capsules and the like. Such a kit is particularly suitable for administering different dosage forms, for example, oral and parenteral dosage forms, for administering the separate compositions at different dosage intervals, or for titrating the separate compositions against one another. To assist compliance, the kit typically comprises directions for administration and may be provided with a so-called memory aid.

### XII. Working Examples

The following example illustrates preparing the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine. A suspension of 2- {3- [4-(2,2-Difluoro-3 -oxo-3,4-dihydro-2H-benzo [1,4] oxazin-6-ylamino)5-fluoro-pyrimidin-2-ylamino]phenoxy}N-methyl-acetamide (1.18 kg, 2.49 mmol, 1 equiv) in methyl ethyl ketone (MEK) (23.6 L, 20 ml/g) was heated to 55° C., whereupon water (1.18L, lml/g) was added, resulting in a solution. The solution was passed through a filter for clarification then held at 55° C. for 1 hour. The subsequent addition of a pre-formed spec-free solution of 1-hydroxy-2-naphthoic acid (515 g, 2.74 mol, 1.1 equiv) in MEK (4.72 L, 4 ml/g) resulted in precipitation of a white solid after approximately 10 minutes. The reaction was cooled to ambient temperature, stirred overnight (18 hours) and then cooled to 5° C. for 2 hours before filtration. The filtered solid was washed with MEK (2×2.36 L, 2×2 ml/g) and dried under reduced pressure at 50° C. for 16 hours. The product, 2-{3-[4-(2,2-difluoro-3-oxo-3,4-dihydro-2H-benzo[1,4]oxazin-6-ylamino)5-fluoro-pyrimidin-2-ylamino]phenoxy}N-methyl-acetamide 1-hydroxy-2-napthoic acid salt, was isolated as a white solid (1.32 kg).

When analyzed by conventional proton NMR (300 MHz, d₆-DMSO), the xinafoate salt gives the following spectrum: δ 2.65 (d, J 4.5 Hz, 3H), 4.34, (s, 2H), 6.46-6.52 (m, 1H), 7.10 (t, J 8.0 Hz, 1H), 7.23-7.28 (m, 2H), 7.36-7.41 (m, 2H), 7.45-7.48 (m, 1H), 7.55-7.62 (m, 2H), 7.64-7.71 (m, 1H), 7.73-7.77 (m, 1H), 7.86-7.95 (m, 2H), 8.14 (d, J 4.0 Hz, 1H), 8.26-8.32 (m, 1H), 9.14 (s, 1H), 9.56 (s, 1H), 11.90-11.96 (m, 1 H).

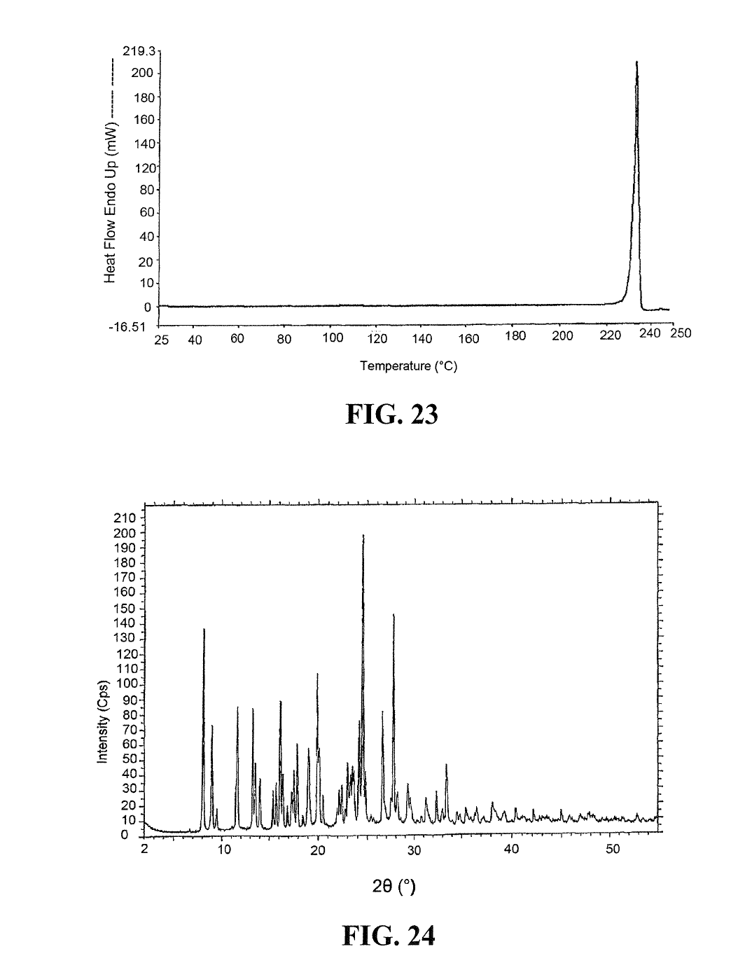

When analyzed by differential scanning calorimetry (DSC) (8.588 mg of the sample was heated from 25 to 250° C. at 20° C. per minute using a Perkin Elmer Diamond DSC with autosampler and a 4-hole side wall vented aluminum pan and lid with nitrogen flow gas), the xinafoate salt shows a sharp endothermic melting peak at 233° C. ±2° C. The DSC trace is shown in FIG. 23.

When characterized by powder X-ray diffraction (PXRD), the xinafoate salt gives the pattern shown in FIG. 24. The characteristic peaks are given in Table 1 below. The main characteristic peaks are at 8.0, 8.9, 11.6, 24.5 and 27.7 degrees two theta (+0.1 degree).

TABLE 1
Characteristic PXRD peaks

| Angle 2-Theta | Relative intensity |
|---|---|
| (degrees) | (%) |
| 8.0 | 68.7 |
| 8.9 | 36.5 |
| 11.6 | 42.6 |
| 13.2 | 42.5 |
| 13.5 | 23.8 |
| 14.0 | 18.7 |
| 15.3 | 15.0 |
| 15.6 | 17.4 |
| 16:1 | 44.5 |
| 16.4 | 20.1 |
| 17.3 | 14.5 |
| 17.5 | 21.4 |
| 17.8 | 30.3 |
| 19.0 | 28.9 |
| 19.8 | 54.0 |
| 20.0 | 28.8 |
| 20.4 | 13.0 |
| 22.1 | 15.0 |
| 22.4 | 16.5 |
| 23.0 | 24.1 |
| 23.2 | 19.9 |
| 23.5 | 22.8 |
| 23.6 | 20.9 |
| 24.1 | 38.1 |
| 24.5 | 100.0 |
| 24.7 | 20.6 |
| 26.6 | 41.1 |
| 27.5 | 12.3 |
| 27.7 | 73.7 |
| 28.1 | 14.1 |
| 29.3 | 16.6 |
| 29.5 | 11.4 |
| 31.2 | 11.8 |
| 32.4 | 14.4 |
| 33.4 | 22.5 |

The powder X-ray diffraction pattern was determined using a Bruker-AXS Ltd D4 powder X-ray diffractometer fitted with an automatic sample changer, a theta-theta goniometer, automatic beam divergence slit, and a PSD Vantec-1 detector. The sample was prepared for analysis by mounting on a low background silicon wafer specimen mount. The specimen was rotated whilst being irradiated with copper K-alphaᵢ X-rays (wavelength=1.5406 Angstroms) with the X-ray tube operated at 40 kV/30 mA. The analyses were performed with the goniometer running in continuous mode set for a 0.2 second count per 0.018° step over a two theta range of 2° to 55°. Peaks were selected manually using Bruker-AXS Ltd evaluation software. The data were collected at 21° C.

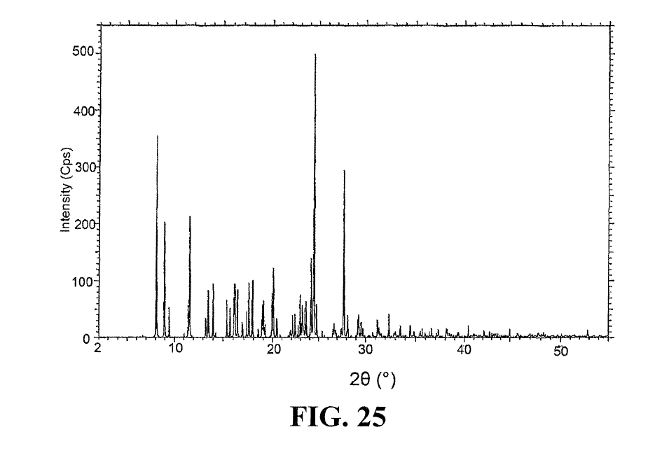

As will be appreciated by a person of ordinary skill in the art, the relative intensities of various peaks may vary due to a number of factors such as for example orientation effects of crystals in the X-ray beam or the purity of the material being analyzed or the degree of crystallinity of the sample. The peak positions may also shift for variations in sample height but the peak positions will remain substantially as stated. A person of ordinary skill in the art will also appreciate that measurements using a different wavelength will result in different shifts according to the Bragg equation −nλ=2d sin θ. Such alternative PXRD patterns generated by use of alternative wavelengths are nevertheless representations of the same material. The main PXRD peaks which have been simulated from a single crystal X-ray analysis are listed in Table 2 below and the corresponding simulated pattern is shown in FIG. 25.

TABLE 2
Characteristic simulated PXRD peaks

| Angle 2-Theta | Relative intensity |
|---|---|
| (degrees) | (%) |
| 8.0 | 72.5 |
| 8.9 | 41.3 |
| 9.4 | 10.5 |
| 11.4 | 11.5 |
| 11.6 | 43.0 |
| 13.5 | 16.6 |
| 14.0 | 19.2 |
| 15.3 | 13.3 |
| 15.7 | 10.2 |
| 16.0 | 14.3 |
| 16.1 | 17.6 |
| 16.4 | 17.1 |
| 17.5 | 19.4 |
| 17.9 | 20.3 |
| 18.9 | 11.7 |
| 19.0 | 13.2 |
| 19.9 | 15.8 |
| 20.1 | 25.1 |
| 23.0 | 15.2 |
| 23.2 | 11.5 |
| 23.5 | 10.2 |
| 23.6 | 12.1 |
| 24.1 | 28.5 |
| 24.4 | 14.1 |
| 24.5 | 100.0 |
| 24.7 | 11.9 |
| 27.7 | 58.5 |

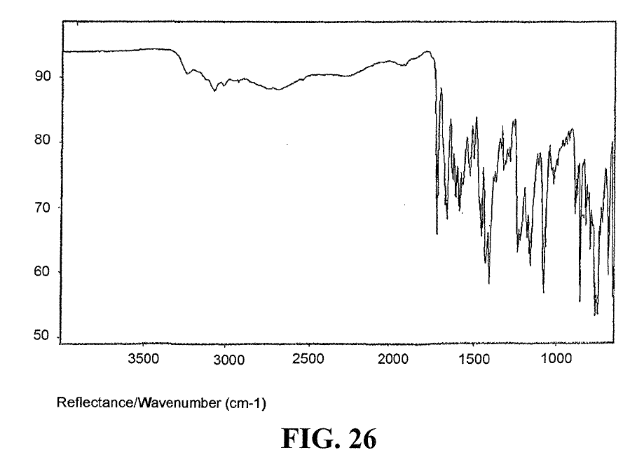

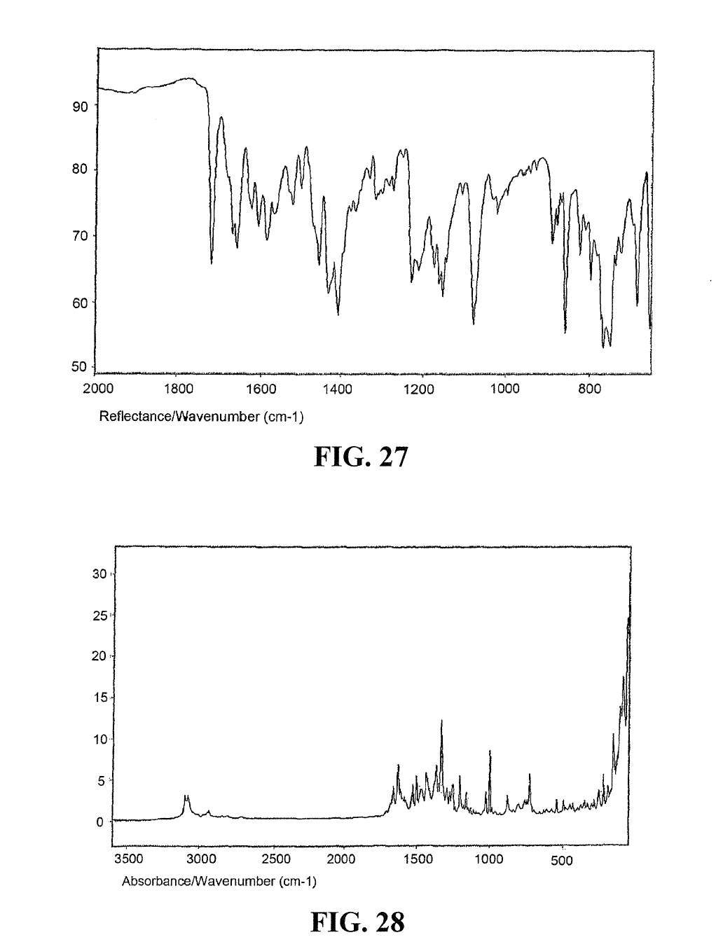

When characterized by Fourier Transform Infra-red (FT-IR) spectroscopy, the xinafoate salt gives the pattern shown in FIG. 26. The fingerprint region is shown in expanded form in FIG. 27. The characteristic peaks are given in Table 3 below (w=weak, s=strong, m=medium). The main characteristic peaks are 1228 (m), 1152 (m), 1078 (s) and 858 (s).

TABLE 3
Characteristic FT-IR peaks
Wavenumber (cm⁻¹)

```
3230* (w)
3069 (w)
3015 (w)
1717 (s)
1669 (m)
1659 (m)
1625 (m)
1608 (m)
1587 (m)
1569 (m)
1523 (m)
1501 (w)
1455 (m)
1431 (s)
1407 (s)
1364 (w)
1331 (w)
1316 (w)
1283 (w)
1272 (w)
1228 (m)
1212 (m)
1174 (m)
1161 (m)
1152 (m)
1107 (w)
1078 (s)
1020 (w)
  928 (w)
  888 (m)
  877 (w)
  858 (s)
  823 (m)
  810 (w)
  796 (m)
  764 (s)
  747 (s)
  734 (w)
  721 (w)
  683 (m)
  653 (m)
```

The FT-IR spectrum was acquired using a ThermoNicolet Nexus FTIR spectrometer equipped with a ‘DurasampllR’ single reflection ATR accessory (diamond surface on zinc selenide substrate) and d-TGS KBr detector. The spectrum was collected at 2 cm⁻¹ resolution and a co-addition of 256 scans for all compounds. Happ-Genzel apodization was used. Because the FT-IR spectrum was recorded using single reflection ATR, no sample preparation was required. Using ATR FT-IR will cause the relative intensities of infrared bands to differ from those seen in a transmission FT-IR spectrum using KBr disc or nujol mull sample preparations. Due to the nature of ATR FT-IR, the bands at lower wavenumber are more intense than those at higher wavenumber. Experimental error, unless otherwise noted, was ±2 cm⁻¹. Peaks were picked using ThermoNicolet Omnic 6.0a software.

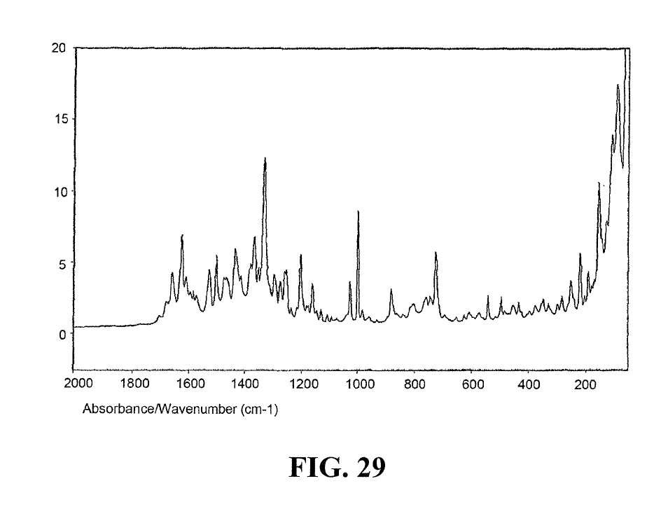

When characterized by Fourier Transform Raman spectroscopy, the xinafoate salt gives the pattern shown in FIG. 28. The fingerprint region is shown in greater detail in FIG. 29. The characteristic peaks are given in Table 4 below (w=weak, s=strong, m=medium). The main characteristic peaks are 1626 (m), 1205 (m), 998 (s), 156 (s) and 91 (S).

TABLE 4
Characteristic FT-Raman peaks
Wavenumber (cm⁻¹)

```
3092 (w)
3071 (w)
1679 (w)
1659 (m)
1626 (m)
1611 (w)
1596 (w)
1584 (w)
1574 (w)
1525 (m)
1502 (m)
1473 (w)
1465 (w)
1434 (m)
1414 (w)
1379 (m)
1365 (m)
1353 (m)
1333 (s)
1296 (m)
1276 (w)
1260 (m)
1253 (rn)
1205 (m)
1162 (w)
1026 (w)
  998 (s)
  879 (w)
  726 (m)
  542 (w)
  495 (w)
  434 (w)
  352 (w)
  332 (w)
  302 (w)
  286 (w)
  253 (w)
  221 (m)
  192 (w)
  156 (s)
  130 (m)
  110 (s)
  91 (s)
  62 (s)
```

The Raman spectrum was collected using a Bruker Vertex70 with Ram 11 module FT-Raman spectrometer equipped with a 1064 nm NdYAG laser and LN-Germanium detector. The spectrum was recorded using 2 cm⁻¹ resolution and Blackman-Harris 4-term apodization. Laser power was 300 mW and 2048 co-added scans were collected. Each sample was placed in a glass vial and exposed to the laser radiation. The data is presented as intensity as a function of Raman shift and is corrected for instrument response and frequency dependent scattering using a white light spectrum from a reference lamp. The Bruker Raman Correct function was used to do the correction. (Bruker software - OPUS 6.0). Experimental error, unless otherwise noted, was ±2 cm⁻¹. Peaks were picked using ThermoNicolet Omnic 6.0a software

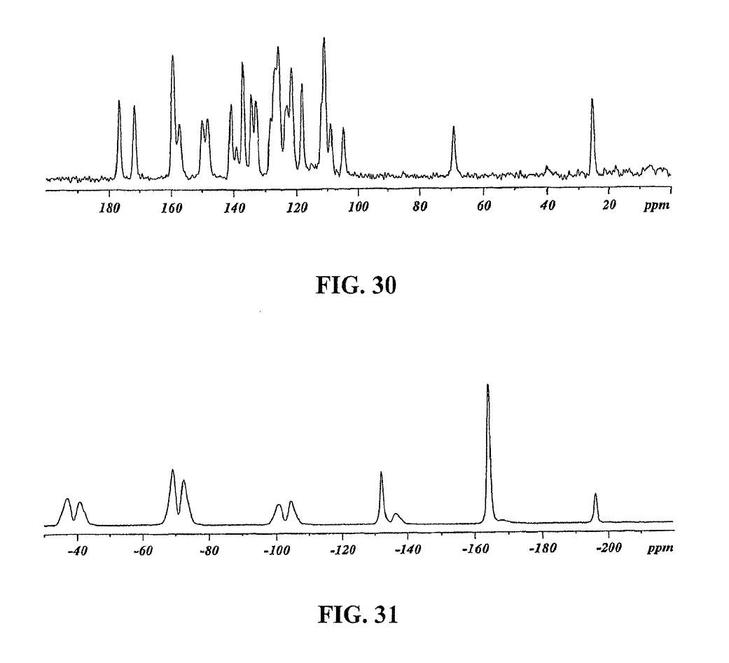

When characterized by proton decoupled ¹³C solid state NMR, the xinafoate salt has the spectrum shown in FIG. 30. The characteristic shifts are given in Table 5 below. The main characteristic shifts are 176.8, 159.4, 137.1, 118.2, 104.9 and 25.4 ppm. Intensities can vary depending on the actual setup of the experimental parameters and the thermal history of the sample and are not therefore necessarily quantitative.

TABLE 5
Characteristic ¹³C solid state NMR shifts

| Chemical shift | |
|---|---|
| (ppm) | Intensity |
| 176.8 | 6.48 |
| 171.8 | 6.04 |
| 159.4 | 10.46 |
| 157.5 | 4.33 |
| 150.0 | 4.66 |
| 148.3 | 4.83 |
| 140.9 | 6.12 |
| 139.2 | 2.37 |
| 137.1 | 9.88 |
| 134.4 | 6.97 |
| 133.1 | 6.41 |
| 128.4 | 4.88 |
| 126.9 | 9.39 |
| 125.8 | 11.22 |
| 123.0 | 6.03 |
| 121.6 | 9.38 |
| 118.2 | 7.96 |
| 110.9 | 12 |
| 109.0 | 4.37 |
| 104.9 | 3.99 |
| 69.3 | 4.01 |
| 25.4 | 6.37 |

Approximately 80 mg of sample were tightly packed into a 4 mm ZrO₂ spinner. The spectrum was collected at ambient conditions on a Bruker-Biospin 4mm BL HFX CPMAS probe positioned into a wide-bore Bruker-Biospin Avance DSX 500 MHz NMR spectrometer. The sample was positioned at the magic angle and spun at 15.0 kHz. The fast spinning speed minimized the intensities of the spinning side bands. The number of scans was adjusted to obtain adequate S/N. The ¹³C solid state spectrum was collected using a proton decoupled cross-polarization magic angle spinning experiment (CPMAS). A proton decoupling field of approximately 85 kHz was applied. 656 scans were collected with the recycle, delay adjusted to 80 seconds. The spectrum was referenced using an external standard of crystalline adamantane, setting its upfield resonance to 29.5 ppm.

When characterized by fluorine solid state NMR, the xinafoate salt has the spectrum shown in FIG. 31. The characteristic shifts are −69.2, −72.4 and −164.0 ppm. Intensities can vary depending on the actual setup of the experimental parameters and the themial history of the sample and are not therefore necessarily quantitative.

The same apparatus was used to acquire the fluorine NMR spectrum as that used to acquire the ¹³C spectrum. The ¹⁹F solid state spectrum was collected using a proton decoupled magic angle spinning (MAS) experiment. The proton decoupling field of approximately 85 kHz was applied and 8 scans were collected. The recycle delay was set to 750 s to ensure acquisition of quantitative spectra. Proton longitudinal relaxation times (¹H T₁) were calculated based on a fluorine detected proton inversion recovery relaxation experiment. Fluorine longitudinal relaxation times (¹⁹F T₁) were calculated based on a fluorine detected fluorine inversion recovery relaxation experiment. The spectrum was referenced using an external sample of trifluoroacetic acid (50% by volume in H₂O), setting its resonance to −76.54 ppm.

Stability:

The present disclosure concerns a xinafoate salt that is suitably stable for use in the disclosed device. The stability of the xinafoate salt may be measured and/or determined using any of the following disclosed methods.

In contrast to the free base, the xinafoate salt of N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine is essentially non-hygroscopic. Hygroscopicity was assessed using dynamic vapor sorption equipment (Surface Measurement Systems Ltd, model DVS-1). The analysis was conducted at 30° C. with a nitrogen gas flow of 200 cc/min. Water sorption and desorption were determined in the range 0 to 90% relative humidity (RH) using 15% RH intervals. Exposure was for a minimum of 2 hours at each humidity, or until the rate of weight change was less than 0.0003%/minute (averaged over 10 minutes). Sample weight was 12.6 mg. The sample was weighed using a CAHN D-200, seven place digital recording balance, which is an integral part of the equipment. The compound showed only 0.6% water sorption at 90% RH. Furthermore, following micronization using jet milling, there was no change in solid form, a negligible decrease in the degree of crystallinity and no significant change in hygroscopicity (0.9% water sorption at 90% relative humidity). Furthermore, the xinafoate salt does not show any hydration or solvation. Solvation/Hydration was assessed by thermogravimetric analysis (TGA) using a TA Instruments Hi-Res TGA 2950 instrument measuring the weight loss of an 8.8 mg sample in an open platinum pan. The sample was heated at 20° C/min from ambient to 300° C. utilizing a nitrogen furnace purge gas. Whereas a single form of the xinafoate salt has hitherto been identified, the free base hydrates to form a hemihydrate and formed a different solvated form in each of nine solvents tested.

In order to test for solid state stability and excipient compatibility, a sample of the xinafoate salt was micronized by jet milling (particle size: D10=0.24 μm; D50=1.15 μm, D90=4.29 μm) and the resulting powder was blended at a 1:100 weight ratio with lactose monohydrate (Respitose grade SV008). Samples were stored for 12 weeks at 25° C./60% relative humidity and 40° C./75% relative humidity and assayed for remaining drug content and impurities at 4, 8 and 12 weeks. The results are shown in Table 6. A control sample was stored at 5° C./0% humidity. In particular disclosed embodiments, the xinafoate salt was stable for at least two years under conditions of 25° C/60% relative humidity, and for at least six months under 40° C./75% relative humidity.

TABLE 6
Stability data

| | % main band | | |
|---|---|---|---|
| | remaining versus control | | |
| Sample | 4 weeks | 8 weeks | 12 weeks |
| API | 100.2 | 99.9 | 100.1 |
| 25° C./60% RH | | | |
| API | 100.4 | 100.0 | 100.0 |
| 40° C./75% RH | | | |
| Blend | 100.2 | 99.9 | 100.1 |
| 25° C./60% RH | | | |
| open vial | | | |
| Blend | 100.4 | 100.0 | 100.0 |
| 40° C./75% RH | | | |
| open vial | | | |
| Blend | 100.3 | 100.4 | 100.4 |
| 40° C./75% RH | | | |
| foil sealed capsule | | | |

The results show that lactose blends of the xinafoate salt have good stability. During the experiment, no change in physical form was detected and no significant degradation was observed.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A device, comprising:
a housing defining a chamber that houses a xinafoate salt derived from N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine for administration through a patient interface located in the housing in communication with the chamber; and
an advancement mechanism capable of distributing the xinafoate salt to the chamber.

2. The device of claim 1 wherein the xinafoate salt is loaded on an elongate carrier.

3. The device of claim 1 wherein the xinafoate salt is a dry powder.

4. The device of claim 3 wherein the dry powder is encapsulated.

5. The device of claim 3 wherein the dry powder is excipient free.

6. The device of claim 1 wherein the xinafoate salt is formulated for administration via inhalation.

7. The device of claim 6 wherein formulated for administration comprises mixing the xinafoate salt with a pharmaceutically acceptable carrier to form a composition suitable for use in the device.

8. The device of claim 7 wherein the xinafoate salt is a dry powder and the pharmaceutically acceptable carrier is lactose.

9. The device of claim 8 wherein the lactose is lactose monohydrate or anhydrous lactose.

10. The device of claim 7 wherein the xinafoate salt is an encapsulated dry powder and the pharmaceutically acceptable carrier is a carbohydrate selected from lactose, dextran, glucose, maltose, sorbitol, xylitol, fructose, sucrose, and trehalose.

11. The device of claim 10 wherein the lactose is lactose monohydrate or anhydrous lactose.

12. The device of claim 7 wherein the composition comprises about 1 to about 20 weight percent of the xinafoate salt and about 99 to about 80 weight percent of the pharmaceutically acceptable carrier.

13. The device of claim 1 wherein the xinafoate salt is a non-hygroscopic xinafoate salt.

14. The device of claim 1 wherein the xinafoate salt is a non-hydrated xinafoate salt

15. The device of claim 1 wherein the xinafoate salt is a non-solvated xinafoate salt.

16. The device of claim 1 wherein the xinafoate salt comprises a particle size suitable for inhalation.

17. The device of claim 16 wherein the mean particle size ranges from about 0.4 μm to about 5 μm.

18. The device of claim 1 wherein the xinafoate salt is a thermodynamically stable crystal.

19. The device of claim 2 wherein the advancement mechanism is operably coupled to the elongate carrier.

20. The device of claim 19 wherein the elongate carrier is loaded with the xinafoate salt.

21. The device of claim 19 wherein the advancement mechanism exposes a predeteimined area of the elongate carrier to air flow during inhalation.

22. The device of claim 19 further comprising a mechanism for applying a force to the elongate carrier to release the xinafoate salt from the elongate carrier.

23. The device of claim 22 wherein the mechanism for applying a force comprises a member positioned to impact, strike, scrape, or brush an exposed area of the elongate carrier.

24. The device of claim 22 wherein the mechanism for applying a force is actuated by inhalation by a patient.

25. The device of claim 22 further comprising a tensioning element for holding an exposed portion of the elongate carrier taut.

26. The device of claim 25 wherein the elongate carrier is a microstructured carrier tape wound on a spool and the advancement mechanism comprises a rotatable winding spool whereby rotation of the winding spool unwinds the microstructured carrier tape which is wound onto the winding spool.

27. The device of claim 26 wherein the microstructured carrier tape comprises a plurality of microdepressions.

28. The device of claim 1 further comprising at least one air inlet in communication with the chamber capable of allowing air flow when a patient inhales through the patient interface.

29. The device of claim 1 further comprising a dosage counter.

30. The device of claim 29 wherein the dosage counter is incorporated into the housing.

31. The device of claim 29 wherein the dosage counter comprises an indicator wheel having indicia indicating dosages dispensed.

32. The device of claim 29 further comprising a dosage counter comprising plural count indicators.

33. The device of claim 29 wherein the dosage counter is an electrical dosage counter.

34. A device, comprising:
a housing defining a chamber that houses a xinafoate salt derived from N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine formulated for administration through a patient interface located in the housing in communication with the chamber;
an inhalation-activatable triggering mechanism that controls dispensation from the source of the xinafoate salt; and
a reset component.

35. The device of claim 34 wherein the triggering mechanism comprises a vane capable of pivotal movement between a closed position and an open position and an activator component that moves between a restrained position and a dispensing position during use.

36. The device of claim 35 wherein the vane is positioned such that inhalation through the patient interface generates an air flow that effectuates the vane's pivotal movement.

37. The device of claim 35 wherein the vane comprises a pivot point that is positioned towards one end of the vane.

38. The device of claim 35 wherein moving the activator component controls dispensation of the xinafoate salt from the source.

39. The device of claim 35 wherein the reset component causes the activator component to move back into its restrained position, which directly or indirectly via one or more intermediate components causes the vane to move from a substantially open position to a closed position.

40. The device of claim 35 wherein the vane and the activator component are arranged to mechanically interengage during a reset cycle.

41. The device of claim 35 wherein the vane is positioned within the patient interface and arranged such that it may be substantially returned to its closed position prior to initiating a reset cycle.

42. The device of claim 41 wherein the vane is positively engaged by a component of the triggering mechanism as the mechanism is reset.

43. The device of claim 35 wherein the triggering mechanism is arranged such that when the activator component is in a restrained position and the vane is closed, the vane mechanically blocks the activator component from moving from its restrained position.

44. The device of claim 43 wherein the vane pivots from a closed position to an open position to allow movement of the activator component, allowing dispensation of the xinafoate salt from the source.

45. The device of claim 43 wherein a blocking component and reset component are positioned at an end of the vane near the vane's pivot point.

46. The device of claim 43 wherein the blocking component and the reset component are a projection.

47. The device of claim 46 wherein the activator component is restrained and the vane is closed, and the projection mechanically engages the activator component.

48. The device of claim 47 wherein the projection is moved out of mechanical engagement with the activator component once the vane is pivoted from its closed to open position, allowing dispensation of the xinafoate salt.

49. The device of claim 48 wherein the reset component moves the activator component from its dispensing position to its restrained position, causing engagement of the projection by the activator component, whereby the vane is closed.

50. The device of claim 35 wherein the triggering mechanism further comprises a catch.

51. The device of claim 50 wherein the catch is pivotally mounted for movement between:
(a) a blocking position in which the catch mechanically prevents the activator component from moving from its restrained position; and
(b) a release position in which it allows the activator component to dispense the xinafoate salt from the source.

52. The device of claim 50 wherein the catch and vane independently comprise an engagable end to allow movement between the catch and the vane.

53. The device of claim 52 wherein the catch further comprises:
a blocking surface to engage the activator component in its restrained position; and
a reset surface which is engaged by the activator component during movement from dispensing to its restrained position.

54. The device of claim 52 wherein the activator component is moved by the reset component, which causes the catch to move back to its blocking position, resulting in the vane being closed.

55. The device of claim 35 wherein the triggering mechanism further comprises a rocker.

56. The device of claim 55 wherein the rocker is mounted for pivotal movement.

57. The device of claim 56 wherein the rocker comprises an end that is engagable with one end of the vane, allowing movement between the vane and the rocker; and a second end engagable with the catch, allowing movement between the rocker and the catch.

58. The device of claim 57 wherein the catch moves the rocker, thereby closing the vane.

59. The device of claim 34 wherein the reset component acts directly on the activator component and moves it into its restrained position.

60. The device of claim 34 wherein the reset component is a projection on a moveable cover.

61. A method, comprising:
using an inhaler to administer a neat xinafoate salt derived from N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine.

62. The method of claim 61 wherein administering comprises administering an effective amount of the xinafoate salt to a subject having a respiratory disorder.

63. The method of claim 61 wherein the xinafoate salt is administered as a dose.

64. The method of claim 63 wherein administration of the dose is accompanied by an audible click.

65. The method of claim 63 wherein the dose is an emitted dose.

66. The method of claim 65 wherein the emitted dose is from about 65% to about 135% of the N4- [(2,2-difluoro-4H-benzo[1,4]oxazin-3-one)-6-yl]-5-fluoro-N2- [3-(methylaminocarbonylmethyleneoxy)phenyl]-2,4-pyrimidinediamine.

67. The method of claim 66 wherein the emitted dose is from about 75% to about 125% of N4- [(2,2-difluoro-4H-benzo[1,4]oxazin-3 -one)-6-yl]-5 -fluoro-N2- [3-(methylaminocarbonylmethyleneoxy)phenyl]-2,4-pyrimidinediamine.

68. The method of claim 65 wherein the dose ranges from about 0.005 mg to about 20 mg.

69. The method of claim 68 wherein the dose corresponds to a fill weight ranging from about 0.1 mg to about 30 mg.

70. The method of claim 69 wherein the fill weight is from about 1 mg to about 2 mg

71. A method for making an inhaler, comprising:
providing a xinafoate salt derived from N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine formulated for use in an inhaler;
loading a microstructured carrier tape with the xinafoate salt; and
associating the microstructured carrier tape with the inhaler.

72. The method of claim 71 wherein loading comprises filling a microdepression.

73. The method of claim 71 wherein the inhaler comprises a housing defining a chamber that houses the xinafoate salt, wherein the xinafoate salt is formulated for administration through a patient port located in the housing in communication with the chamber, and an advancement mechanism capable of distributing the xinafoate salt to the chamber.

74. The method of claim 71 wherein the inhaler is a dry powder inhaler.

75. A component for use in an inhaler, comprising:
a xinafoate salt derived from N4-[(2,2-difluoro-4H-benzo[1,4]oxazin-3-one)-6-yl]-5-fluoro-N2-[3-(methylaminocarbonylmethyleneoxy)phenyl]-2,4-pyrimidinediamine; and
at least one microdepression containing the xinafoate salt.

76. The component of claim 75 wherein the component is an elongate carrier.

77. The component of claim 76 wherein the microdepression is selected from a microdimple, a microblister, or a microgroove.

78. The component of claim 76 wherein the microdepression comprises from about 0.1 to about 1 mg of the xinafoate salt.
